SEQUENCE LISTING (61 sequences)

SEQ ID NO: 1 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - protein, 14 aa, synthetic construct
TTGACTTACTGAAG

SEQ ID NO: 3 - protein, 15 aa, synthetic construct
  VARIANT 4: note = X at this position for this sequence is referred to as "v" in the Specification for this sequence, and v is Q, D or E.
  VARIANT 11: note = X at this position for this sequence is referred to as "w" in the Specification for this sequence, and w is S or N.
  VARIANT 12: note = X at this position for this sequence is referred to as "x" in theSpecification for this sequence, and x is I, H, N, or I.
  VARIANT 13: note = X at this position for this sequence is referred to as "y" in the Specification for this sequence, and y is D, A, I, N, H, K, S, G, or null.
  VARIANT 14: note = X at this position for this sequence is referred to as "z" in the Specification for this sequence, and z can be GGRPALE, GGKQALE, GGKQALETVQRLLPVLCQDHG, GGKQALETVQRLLPVLCQAHG, GKQALETVQRLLPVLCQDHG, GKQALETVQRLLPVLCQAHG, GGKQALETVQRLLPVLCQD, or GGKQALETVQRLLPVLCQA.
  VARIANT 15: note = X at this position in the sequence is referred to as "alpha" in the Specification for this sequence, and alpha is four consecutive amino acids.
LTPXQVVAIAXXXXX

SEQ ID NO: 4 - protein, 7 aa, synthetic construct
GGRPALE

SEQ ID NO: 5 - protein, 7 aa, synthetic construct
GGKQALE

SEQ ID NO: 6 - protein, 21 aa, synthetic construct
GGKQALETVQRLLPVLCQDHG

SEQ ID NO: 7 - protein, 21 aa, synthetic construct
GGKQALETVQRLLPVLCQAHG

SEQ ID NO: 8 - protein, 20 aa, synthetic construct
GKQALETVQRLLPVLCQDHG

SEQ ID NO: 9 - protein, 20 aa, synthetic construct
GKQALETVQRLLPVLCQAHG

SEQ ID NO: 10 - protein, 19 aa, synthetic construct
GGKQALETVQRLLPVLCQD

SEQ ID NO: 11 - protein, 19 aa, synthetic construct
GGKQALETVQRLLPVLCQA

SEQ ID NO: 12 - protein, 4 aa, synthetic construct
GHGG

SEQ ID NO: 13 - protein, 4 aa, synthetic construct
HGSG

SEQ ID NO: 14 - protein, 4 aa, synthetic construct
HGGG

SEQ ID NO: 15 - protein, 4 aa, synthetic construct
GGHD

SEQ ID NO: 16 - protein, 4 aa, synthetic construct
GAHD

SEQ ID NO: 17 - protein, 4 aa, synthetic construct
AHDG

SEQ ID NO: 18 - protein, 4 aa, synthetic construct
PHDG

SEQ ID NO: 19 - protein, 4 aa, synthetic construct
GPHD

SEQ ID NO: 20 - protein, 4 aa, synthetic construct
GHGP

SEQ ID NO: 21 - protein, 4 aa, synthetic construct
PHGG

SEQ ID NO: 22 - protein, 4 aa, synthetic construct
PHGP

SEQ ID NO: 23 - protein, 4 aa, synthetic construct
AHGA

SEQ ID NO: 24 - protein, 4 aa, synthetic construct
LHGA

SEQ ID NO: 25 - protein, 4 aa, synthetic construct
VHGA

SEQ ID NO: 26 - protein, 4 aa, synthetic construct
IVHG

SEQ ID NO: 27 - protein, 4 aa, synthetic construct
IHGM

SEQ ID NO: 28 - protein, 4 aa, synthetic construct
RHGD

SEQ ID NO: 29 - protein, 4 aa, synthetic construct
RDHG

SEQ ID NO: 30 - protein, 4 aa, synthetic construct
RHGE

SEQ ID NO: 31 - protein, 4 aa, synthetic construct
HRGE

SEQ ID NO: 32 - protein, 4 aa, synthetic construct
RHGD

SEQ ID NO: 33 - protein, 4 aa, synthetic construct
HRGD

SEQ ID NO: 34 - protein, 4 aa, synthetic construct
GPYE

SEQ ID NO: 35 - protein, 4 aa, synthetic construct
NHGG

SEQ ID NO: 36 - protein, 4 aa, synthetic construct
THGG

SEQ ID NO: 37 - protein, 4 aa, synthetic construct
GTHG

SEQ ID NO: 38 - protein, 4 aa, synthetic construct
GSGS

SEQ ID NO: 39 - protein, 4 aa, synthetic construct
GSGG

SEQ ID NO: 40 - protein, 4 aa, synthetic construct
GGGG

SEQ ID NO: 41 - protein, 4 aa, synthetic construct
GRGG

SEQ ID NO: 42 - protein, 4 aa, synthetic construct
GKGG

SEQ ID NO: 43 - protein, 36 aa, synthetic construct
  VARIANT 12: note = X at this position for this sequence is referred to in the Specification as *RVD* and corresponds to the first peptide x of dipeptide xy of SEQ ID NO:3, here x is I, H, or N.
  VARIANT 13: note = X at this position for this sequence is referred to in the Specification as *RVD* and corresponds to the second peptide y of dipeptide xy of SEQ ID NO:3, here y is D, A, I, N, H, K, S, G, or null.
LTPEQVVAIASXXGGKQALETVQRLLPVLCQAGHGG

SEQ ID NO: 44 - DNA, 810 bp, synthetic construct
  source 1..810: mol_type = other DNA
agaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtccca

SEQ ID NO: 45 - DNA, 37 bp, synthetic construct
  source 1..37: mol_type = other DNA
cagagaaaggctcttaaaaatgcagcgcaatctccag

SEQ ID NO: 46 - DNA, 43 bp, synthetic construct
  source 1..43: mol_type = other DNA
cacttaactatcttgggctgtgacaaagtcacatggttcacac

SEQ ID NO: 47 - DNA, 43 bp, synthetic construct
  source 1..43: mol_type = other DNA
gtgtgaaccatgtgactttgtcacagcccaagatagttaagtg

SEQ ID NO: 48 - DNA, 39 bp, synthetic construct
  source 1..39: mol_type = other DNA
ctgatctagaacttactgtgaatccttgtcacctgccac

SEQ ID NO: 49 - DNA, 38 bp, synthetic construct
  source 1..38: mol_type = other DNA
gataagaaggaggccagcacgataggaacatatgacac

SEQ ID NO: 50 - DNA, 18 bp, synthetic construct
  source 1..18: mol_type = other DNA
tcatccatccgacattga

SEQ ID NO: 51 - DNA, 16 bp, synthetic construct
  source 1..16: mol_type = other DNA
agttgacttactgaag

SEQ ID NO: 52 - DNA, 18 bp, synthetic construct
  source 1..18: mol_type = other DNA
aatggagagagaattgaa

SEQ ID NO: 53 - DNA, 96 bp, synthetic construct
  source 1..96: mol_type = other DNA
tgaattgctatgtgtctgggtttcatccatccgacattgaagttgacttactgaagaatggagagagaattgaaaaagtggagcattcagacttgt

SEQ ID NO: 54 - DNA, 30 bp, synthetic construct
  source 1..30: mol_type = other DNA
acattgaagttgacttactgaagaatggag

SEQ ID NO: 55 - DNA, 16 bp, synthetic construct
  source 1..16: mol_type = other DNA
acattgaagaatggag

SEQ ID NO: 56 - protein, 23 aa, synthetic construct
GKGRKYFHHNSDFHIPKATLKDS

SEQ ID NO: 57 - protein, 23 aa, synthetic construct
GKDRKYFHHNSDFYIPKATLKDS

SEQ ID NO: 58 - protein, 23 aa, synthetic construct
KDSGSYFCRGLFGSKNVSSETVN

SEQ ID NO: 59 - protein, 23 aa, synthetic construct
KDSGSYFCRGLVGSKNVSSETVN

SEQ ID NO: 60 - protein, 22 aa, synthetic construct
MSRSVALAVLALLSLSGLEAIQ

SEQ ID NO: 61 - DNA, 92 bp, synthetic construct
  source 1..92: mol_type = other DNA
atgtctcgctccgtggccttagctgtgctcgcgctactctctctttctggcctggaggctatccagcgtgagtctctcctaccctcccgctc

(19) United States
(12) Patent Application Publication
(10) Pub. No.: US 2024/0182856 A1
(43) Pub. Date: Jun. 6, 2024

(54) ENGINEERED IMMUNE CELL THERAPIES

(71) Applicant: Factor Bioscience Inc., Cambridge, MA (US)

(72) Inventors: Matthew ANGEL, Cambridge, MA (US); Christopher ROHDE, Cambridge, MA (US); Mitchell KOPACZ, Cambridge, MA (US); Jasmine HARRIS, Cambridge, MA (US); James PAN, Cambridge, MA (US)

(21) Appl. No.: 18/461,368

(22) Filed: Sep. 5, 2023

Related U.S. Application Data
(63) Continuation of application No. PCT/US2022/019020, filed on Mar. 4, 2022.
(60) Provisional application No. 63/157,332, filed on Mar. 5, 2021.

(51) Int. Cl. C12N 5/0783; C12N 15/10

(52) U.S. Cl. CPC C12N 5/0646; C12N 15/102; C12N 2506/1307; C12N 2510/00

(57) ABSTRACT

The present disclosure relates in part to engineered immune cells that are, inter alia, silenced from a host immune response.

### CROSS-REFERENCE TO RELATED APPLICATION

This application is a continuation of International Patent Application No. PCT/US2022/019020, filed on Mar. 4, 2022, which claims the benefit of U.S. Provisional Patent Application No. 63/157,332, filed Mar. 5, 2021, the entire contents of the aforementioned patent applications are incorporated herein by reference.

### FIELD

The present disclosure relates to engineered immune cells that evade recognition and/or clearance by a host immune system.

### SEQUENCE LISTING

The application contains a Sequence Listing which has been submitted electronically in XML file format and is hereby incorporated by reference in its entirety. Said XML copy, created on Sep. 5, 2023, is named 61057-717.301.xml and is 59,333 bytes in size.

### BACKGROUND

Autologous engineered cell therapies such as autologous chimeric antigen receptor T-cell (CAR-T) therapies have revolutionized the treatment of hematologic cancers, however they are limited by manufacturing time and variability, the requirement for lymphodepletion, and side effects related to cytokine release. Allogeneic cell therapies derived from gene-edited induced pluripotent stem cells (iPSCs) are being developed to address the challenges associated with autologous engineered cell therapies. These “off-the-shelf” cell therapies contain specific edits designed to reduce immune rejection and to confer enhanced therapeutic properties and greater safety. However, efficient, footprint-free, biallelic targeting of defined loci in iPSCs remains technically challenging with current gene-editing approaches.

Further, while induced pluripotent stem cells (iPSCs) readily differentiate into a wide variety of cell types both in vitro and in vivo, the development of directed differentiation protocols that reliably yield pure populations of functional cells has proved challenging, in particular when differentiating into cell of the lymphoid or myeloid lineage. Generating functional immune cells from iPSCs is of particular interest to support the development of off-the-shelf engineered cell therapies for immune-oncology applications.

What is needed is improved compositions and methods for generating cellular therapies that can be engineered and produced in a practical manner.

### SUMMARY

Accordingly, the present disclosure relates to compositions and methods for cellular therapies, e.g., engineered immune cells that evade recognition and/or clearance by a host immune system and therefore have a therapeutic effect that is not reduced or ablated by a subject's immune response.

In one aspect, there is provided an isolated immune cell comprising a genetically engineered disruption in a beta-2-microglobulin (B2M) gene, e.g., a loss of function, optionally in both alleles, of the B2M gene, wherein the immune cell is selected from a lymphoid cell or a myeloid cell. In some cases the lymphoid cell is a T cell, e.g., a cytotoxic T cell or gamma-delta T cell; an NK cell; or an NK-T cell. In some cases, the myeloid cell is a macrophage, e.g., an M1 macrophage or an M2 macrophage. In embodiments, the immune cell has downregulated MHC class I expression and/or activity. In embodiments, the immune cell has reduced or eliminated susceptibility to cell killing by T cells or other immune cells as compared to another immune cell which comprises a genetically engineered disruption in the B2M gene. In embodiments, the immune cell has reduced or eliminated immune cell fratricide, e.g., NK-cell fratricide. In embodiments, the immune cell is a stealth cell, e.g., the immune cell is not substantially recognized by an immune system upon administration to a subject.

In some cases, the immune cell expresses a fusion protein comprising a B2M polypeptide and a HLA-A, HLA-B, HLA-C, HLA-E, HLA-F and HLA-G polypeptide. The fusion protein may be expressed by insertion of a repair template into a single or double strand break of the B2M gene; in some cases, the repair template comprises the coding sequence for B2M and the HLA gene.

Notably, the fusion protein replaces endogenous B2M and HLA pairs expressed by an immune cell, thereby reducing the likelihood that the immune cell will be reduced or eliminated by a host immune cell.

In embodiments, the immune cell, optionally a T cell or NK cell, is genetically modified to express a recombinant chimeric antigen receptor (CAR) comprising an intracellular signaling domain, a transmembrane domain, and an extracellular domain comprising an antigen binding region. In embodiments, the immune cell, optionally a T cell or NK cell, is engineered to be directed to ROR1 and/or CD19.

In embodiments, the present cells, e.g., B2M knockout immune cell, e.g., T cells, NK cells, or macrophages, have substantially reduced or ablated self-kill activity and, rather, self-activation activity (even in the absence of cytokines like IL-2 and IL-15). Further, the present cells, e.g., B2M knockout immune, e.g., T cells, NK cells, or macrophages have tumoricidal activity (even in the absence of cytokines like IL-2 and IL-15) and have unexpected expansion and proliferation properties.

In another aspect, there is provided a method of making an engineered immune cell, comprising (a) reprogramming a somatic cell to an iPS cell, the reprogramming comprising contacting the iPS cell with a ribonucleic acid (RNA) encoding one or more reprogramming factors; (b) disrupting a B2M gene in the iPS cell, the disrupting comprising gene-editing the cell by contacting the cell with RNA encoding one or more gene-editing proteins; and (c) differentiating the iPS cell into an immune cell, wherein the immune cell is selected from a lymphoid cell or a myeloid cell. In some cases the lymphoid cell is a T cell, e.g., a cytotoxic T cell or gamma-delta T cell; an NK cell; or an NK-T cell. In some cases, the myeloid cell is a macrophage, e.g., an M1 macrophage or an M2 macrophage.

In another aspect, there is provided a method of treating cancer, comprising (a) obtaining an isolated immune cell comprising a genetically engineered disruption in a B2M gene; and (b) administering the isolated immune cell to a subject in need thereof, wherein the immune cell is selected from a lymphoid cell or a myeloid cell. In some cases the lymphoid cell is a T cell, e.g., a cytotoxic T cell or gamma-delta T cell; an NK cell; or an NK-T cell. In some cases, the myeloid cell is a macrophage, e.g., an M1 macrophage or an M2 macrophage. The immune cell may be further genetically engineered to express a chimeric antigen receptor (CAR).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. The drawings and description are to be regarded as illustrative in nature, and not as restrictive. Any description herein concerning a specific composition and/or method apply to and may be used for any other specific composition and/or method as disclosed herein. Additionally, any composition disclosed herein is applicable to any herein-disclosed method. In other words, any aspect or embodiment described herein can be combined with any other aspect or embodiment as disclosed herein.

### BRIEF DESCRIPTION OF THE DRAWINGS

FIG. 1A shows a non-limiting schematic of the mRNA-based reprogramming and gene-editing, followed by differentiation of the present disclosure. FIG. 1B illustrates differentiated cells killing cancer cells.

FIG. 2 shows the design of the gene-editing scheme for beta-2-microglobulin (B2M); shown are the following sequences: TCATCCATCCGACATTGA (SEQ ID NO: 50), AGTTGACTTACTGAAG (SEQ ID NO: 51), AATGGAGAGAGAATTGAA (SEQ ID NO: 52).

FIG. 3 shows an RNA gel demonstrating gene-editing of B2M.

FIG. 4 shows a sequencing experiment that shows the 14 base pair deletion from a gene-edited B2M; shown are the following sequences from bottom to top: ACATTGAAGAATGGAG (SEQ ID NO: 55), ACATTGAAGTTGACTTACTGAAGAATGGAG (SEQ ID NO: 54), and TGAATTGCTATGTGTCTGGGTTTCATCCATCCGACATTGAAGTTGACTTACTGAAGA ATGGAGAGAGAATTGAAAAAGTGGAGCATTCAGACTTGT (SEQ ID NO: 53).

FIG. 5 shows RNA levels of B2M with or without IFN gamma activation (“IFNY”; two left bars are the B2M knockout and the two right bars are näive cells).

FIG. 6 shows a sequencing experiment that demonstrates heterozygosity of CD16a (at G147D dbSNP:rs443082, Y158H dbSNP:rs396716, and F176V dbSNP:rs396991); shown are the following sequences from top to bottom: GKGRKYFHHNSDFHIPKATLKDS (SEQ ID NO: 56), GKDRKYFHHNSDFYIPKATLKDS (SEQ ID NO: 57), KDSGSYFCRGLFGSKNVSSETVN (SEQ ID NO: 58), and KDSGSYFCRGLVGSKNVSSETVN (SEQ ID NO: 59).

FIG. 7A-B shows images of control (PMBC-isolated) NK cells in co-culture with K-562 tumor cells, demonstrating NK Cell cytotoxicity of tumor cell (note immunothrombosis or “clumping”).

FIG. 8A-B shows images of the gene edited and differentiated cells of the present disclosure (e.g., B2M knockout NK cells) in co-culture with K-562 tumor cells, demonstrating NK Cell cytotoxicity of tumor cell (note immunothrombosis or “clumping”).

FIG. 9A-FIG. 9H show results of the cytokine release assay with the Luminex MAGPIX. Unless indicated (i.e. “+IL2, IL15”), conditions are without added IL-2 or IL-15. Further, ratio of cells are indicated (1:1 or 3:1). As elsewhere herein, PBMC-NK are control NK cells. FIG. 9A shows interferon gamma. FIG. 9B shows IL-2. FIG. 9C shows IL-7. FIG. 9D shows IL-13. FIG. 9E shows MIP-1a. FIG. 9F shows MIP-1b. FIG. 9G shows TNFa. FIG. 9H shows GM-CSF.

FIG. 10A-FIG. 10D show flow cytometry data for a gene edited and differentiated cells of the present disclosure (e.g., B2M knockout NK cells) as described in the Examples.

FIG. 11A shows the structure for the B2M-HLA-E repair template. FIG. 11B shows an ideal target site for the B2M-HLA-A repair template is shown (SEQ ID NO: 60: MSRSVALAVLALLSLSGLEAIQ; and SEQ ID NO: 61 ATGTCTCGCTCCGTGGCCTTAGCTGTGCTCGCGCTACTCTCTCTTTCTGGCCTGGAGG CTATCCAGCgtgagtctctcctaccctcccgctc). FIG. 11C shows additional target binding sites (SEQ ID NO: 60 and SEQ ID NO: 61 are again shown). FIG. 11D shows a gel with sizes of two lines having the B2M-HLA-E repair template inserted. FIG. 11E includes graphs showing the intensities of signal and ratios thereof from the bands shown in FIG. 11D. FIG. 11F shows a gel with sizes of two lines having the B2M-HLA-E repair template inserted. FIG. 11G includes graphs showing the intensities of signal and ratios thereof from the bands shown in FIG. 11F.

FIG. 11H shows relevant sequences in the B2M-HLA-E repair template.

FIG. 12A and FIG. 12B, show target site sequences and repair templates for replacing the phenylalanine (F) at position 158 of CD16a with a valine (V). Relevant sequences are shown in these figures.

### DETAILED DESCRIPTION

The present disclosure is based, in part, on the discovery that immune cells, of the lymphoid cell or myeloid lineage, e.g., T cells, NK cells, and macrophages, can be gene-edited and differentiated, using mRNA- and iPS-based methods, to yield therapeutic cells that are immune silenced, yet self-activating, proliferative, and anti-tumoral.

Cytotoxic lymphocytes, including T cells and NK cells, are being developed as allogeneic, “off-the-shelf”, cell therapies for the treatment of hematological and solid tumors. Allogenic lymphocyte therapies face challenges, however, including limited expansion potential and limited in vivo persistence due to host immune rejection. To address these challenges, the present disclosure relates, in part, to methods for manufacturing mRNA-reprogrammed iPSC lines with a biallelic knockouts of the beta-2 microglobulin (B2M) gene, a key component of MHC class I molecules, using an mRNA-encoded chromatin context-sensitive gene-editing endonuclease. As disclosed herein, these B2M-knockout iPSCs were then differentiated using a novel, fully suspension process that replaces specialized micropatterned culture vessels with a spheroid culture step. The resulting lymphocytes were characterized for surface markers via flow cytometry and incubated with cancer cells to assess tumor cell engagement and cytotoxicity. Notably, consistently higher yields of lymphocytes were obtained from the B2M-knockout iPSC line relative to a parental, wild-type iPSC line. Both wild-type and B2M-knockout lymphocytes cells killed 75-90% of K562 cells after 24 hours (effector to target (E:T) ratio of 5:1). Interestingly, cytotoxic lymphocytes derived from B2M-knockout iPSCs exhibited greater K562 cell killing with the addition of IL15 and IL2, while killing by wild-type cells was not controlled by these activating cytokines. Cancer cell killing activity was maintained through cryopreservation, albeit at a reduced level (15-40% reduction in activity). Accordingly, B2M-knockout iPSCs of the present disclosure are an ideal source of cytotoxic lymphocytes for the development of “off-the-shelf” allogeneic cell therapies for the treatment of cancer and without substantial host immune rejection.

In one aspect, there is provided an isolated immune cell comprising a genetically engineered disruption in a beta-2-microglobulin (B2M) gene, e.g., a loss of function, optionally in both alleles, of the B2M gene, wherein the immune cell is selected from a lymphoid cell or a myeloid cell. In some cases the lymphoid cell is a T cell, e.g., a cytotoxic T cell or gamma-delta T cell; an NK cell; or an NK-T cell. In some cases, the myeloid cell is a macrophage, e.g., an M1 macrophage or an M2 macrophage. In embodiments the immune cell is a NK cell.

The present immune cell is sometimes referred to herein as an “engineered immune cell”.

In another aspect, there is provided a method of making an engineered immune cell, comprising (a) reprogramming a somatic cell to an iPS cell, the reprogramming comprising contacting the iPS cell with a ribonucleic acid (RNA) encoding one or more reprogramming factors; (b) disrupting a B2M gene in the iPS cell, the disrupting comprising gene-editing the cell by contacting the cell with RNA encoding one or more gene-editing proteins; and (c) differentiating the iPS cell into an immune cell, wherein the immune cell is selected from a lymphoid cell or a myeloid cell. In some cases the lymphoid cell is a T cell, e.g., a cytotoxic T cell or gamma-delta T cell; an NK cell; or an NK-T cell. In some cases, the myeloid cell is a macrophage, e.g., an M1 macrophage or an M2 macrophage.

In another aspect, there is provided a method of treating cancer, comprising (a) obtaining an isolated immune cell comprising a genetically engineered disruption in a B2M gene; and (b) administering the isolated immune cell to a subject in need thereof, wherein the immune cell is selected from a lymphoid cell or a myeloid cell. In some cases the lymphoid cell is a T cell, e.g., a cytotoxic T cell or gamma-delta T cell; an NK cell; or an NK-T cell. In some cases, the myeloid cell is a macrophage, e.g., an M1 macrophage or an M2 macrophage.

Immune Silencing

In embodiments, the present immune cell is engineered to evade recognition and/or clearance by a host immune system. In embodiments, the present immune cell is a stealth immune cell. In embodiments, the present immune cell is not substantially recognized by an immune system upon administration to a subject.

In embodiments, the present immune cell has reduced or eliminated susceptibility to cell killing by T cells as compared to an immune cell which does not comprise a genetically engineered disruption in the B2M gene. In embodiments, the present immune cell has reduced or eliminated susceptibility to cell killing by other immune cells as compared to another immune cell which comprises a genetically engineered disruption in the B2M gene.

In embodiments, the present immune cell is characterized in that the expression of B2M is reduced or inhibited. In embodiments, the present immune cell is characterized in that the function of B2M is reduced or inhibited.

In embodiments, the present immune cell is characterized in that the expression of MHC class I is reduced or inhibited. In embodiments, the present immune cell is characterized in that the function of MHC class I is reduced or inhibited.

In embodiments, the B2M gene is a human B2M gene (e.g., NCBI Reference Sequence: NG_012920). The sequence of the B2M gene of various embodiments is provided in the EXAMPLES section herein. B2M, is the light chain of MHC class I molecules, and as such an integral part of the major histocompatibility complex In human, B2M is encoded by the b2m gene which is located on chromosome 15. The human protein is composed of 119 amino acids and has a molecular weight of 11.8 kilodaltons (e.g., UniProtKB-P61769). The amino acid sequence of human beta-2-microglobulin (B2M) is:

(SEQ ID NO: 1)
MSRSVALAVLALLSLSGLEAIQRTPKIQVYSRHPAENGKSNFLNCY
VSGFHPSDIEVDLLKNGERIEKVEHSDLSFSKDWSFYLLYYTEFTP
TEKDEYACRVNHVTLSQPKIVKWDRDM.

In embodiments, the present immune cell has genetically engineered disruptions of all substantially all copies of the B2M gene. In embodiments, the present immune cell has a loss of function of the B2M gene. In embodiments, the present immune cell has a loss of function of both alleles of the B2M gene.

In embodiments, the genetically engineered disruption of the B2M gene is in exon 3 of human B2M. In embodiments, the genetically engineered disruption of the B2M gene is a deletion. In embodiments, the deletion is about 10 to about 20 nucleotides. In embodiments, the deletion is near nucleotides 500 to 550 of the human B2M gene. In embodiments, the deletion is of the sequence TTGACTTACTGAAG (SEQ ID NO: 2), or a functional equivalent thereof.

In embodiments, the present immune cell has downregulated MHC class I expression and/or activity.

In embodiments, the genetically engineered disruption of B2M comprises a gene-edit and the gene-edit is caused by contacting the cell with RNA encoding one or more gene-editing proteins.

In embodiments, the present immune cell is engineered to be further immune silenced, e.g., in addition to B2M (MHC Class I) disruption. In embodiments, the present immune cell is engineered to be disrupted at the human MHC II transactivator (CIITA) gene (NCBI Reference Sequence: NG_009628.1).

In embodiments, the present immune cell has downregulated MHC class II expression and/or activity.

In embodiments, the present immune cell is characterized in that the expression of CIITA is reduced or inhibited. In embodiments, the present immune cell is characterized in that the function of CIITA is reduced or inhibited.

In embodiments, the present immune cell is characterized in that the expression of MHC class II is reduced or inhibited. In embodiments, the present immune cell is characterized in that the function of MHC class II is reduced or inhibited.

In embodiments, the genetically engineered disruption of CIITA comprises a gene-edit and the gene-edit is caused by contacting the cell with RNA encoding one or more gene-editing proteins.

In embodiments, the present immune cell is characterized in that the expression of B2M and CIITA is reduced or inhibited. In embodiments, the present immune cell is characterized in that the function of B2M and CIITA is reduced or inhibited.

In embodiments, the present immune cell is characterized in that the expression of MHC class I and MHC class II are reduced or inhibited. In embodiments, the present immune cell is characterized in that the function of MHC class I and MHC class II are reduced or inhibited.

In embodiments, the genetically engineered disruption of B2M and CIITA comprises a gene-edit and the gene-edit is caused by contacting the cell with RNA encoding one or more gene-editing proteins.

In embodiments, the present immune cell comprises a genetically engineered alteration in one or more genes selected from HLA-A, HLA-B, HLA-C, HLA-E, HLA-F and HLA-G.

In embodiments, the immune cell expresses a fusion protein comprising a B2M polypeptide and a HLA-A, HLA-B, HLA-C, HLA-E, HLA-F and HLA-G polypeptide. The fusion protein may be expressed by insertion of a repair template into a single or double strand break of the B2M gene; in some cases, the repair template comprises the coding sequence for B2M and the HLA gene. Notably, the fusion protein replaces endogenous B2M and HLA pairs expressed by an immune cell, thereby reducing the likelihood that the immune cell will be reduced or eliminated by a host immune cell.

In embodiments, the present immune cell does not comprise a genetically engineered alteration in one or more genes selected from HLA-A, HLA-B, HLA-C, HLA-E, HLA-F and HLA-G.

In embodiments, the genetically engineered alteration is a genetically engineered reduction or elimination in expression and/or activity of one or more genes selected from HLA-A, HLA-B, HLA-C, HLA-E, HLA-F and HLA-G.

In embodiments, the genetically engineered alteration is a genetically engineered increase in expression and/or activity of one or more genes selected from HLA-A, HLA-B, HLA-C, HLA-E, HLA-F and HLA-G.

In embodiments, the genetically engineered disruption of B2M is combined with a genetically engineered expression of a fusion between B2M or a fragment thereof and one or more genes and/or fragments thereof selected from HLA-A, HLA-B, HLA-C, HLA-E, HLA-F and HLA-G.

In embodiments, the B2M or fragment thereof and one or more genes and/or fragments thereof are separated by a linker region. In embodiments the linker is (G4S)₃.

In embodiments, the genetically engineered alteration is a genetically engineered increase in expression and/or activity of one or more genes selected from IL-2, IL-15, IL-21. In embodiments, the IL-15 contains the N72D mutation. In embodiments, the IL-15 is fused to the cytokine binding domain of IL-15Rα.

In embodiments, the present immune cell is characterized in that the expression of negative regulators of IL-15 signaling are reduced or inhibited. In embodiments, the negative regulator of IL-15 signaling is the CISH protein. In embodiments, the reduction or inhibition of negative regulators of IL-15 signaling is achieved by genetically engineered disruption of the CISH gene. The Cytokine-inducible SH2-containing protein (CISH) gene is found at gene ID:NG_023194.1.

In embodiments, the genetically engineered disruption of CISH comprises a gene-edit and the gene-edit is caused by contacting the cell with RNA encoding one or more gene-editing proteins.

Immune Cells

In embodiments, the present immune cell is of the lymphoid cell lineage or the myeloid cell lineage.

In some cases, the lymphoid cell is a T cell, e.g., a cytotoxic T cell or gamma-delta T cell.

In some cases, the lymphoid cell is an NK cell, e.g., an NK-T cell. The NK cell may be a human cell.

In some cases, the myeloid cell is a macrophage, e.g., an M1 macrophage or an M2 macrophage.

In various embodiment, the immune cell is reprogrammed from a stem cell, e.g., an iPSC, and differentiated into the immune cell.

In embodiments, the immune cell has a disruption in its beta-2-microglobulin (B2M) gene.

In embodiments, the immune cell has a disruption in its beta-2-microglobulin (B2M) gene and expresses a fusion protein comprising a B2M polypeptide and an HLA polypeptide (e.g., a HLA-A, HLA-B, HLA-C, HLA-E, HLA-F and HLA-G polypeptide).

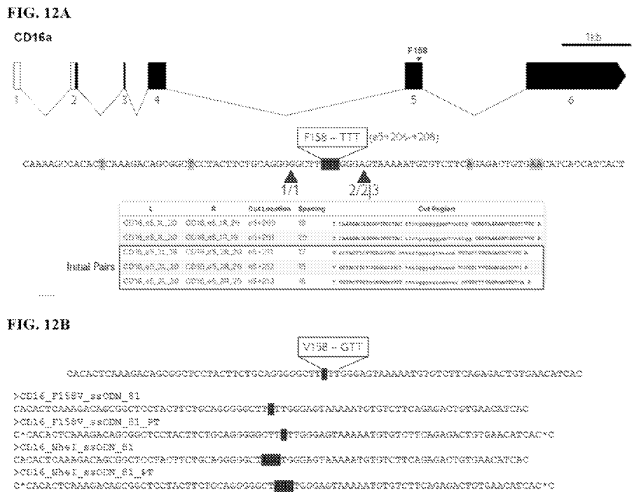

In embodiments, the immune cell is gene edited to express a high affinity variant of CD16a (See, FIG. 12A and FIG. 12B).

In embodiments, the myeloid cell is a cell derived from, or derivable from, a common myeloid progenitor cell. In embodiments, the myeloid cell is a megakaryocyte, erythrocyte, mast cell, or myeloblast. In embodiments, the myeloid cell is a cell derived from, or derivable from, a myeloblast. In embodiments, the myeloid cell is a basophil, neutrophil, eosinophil, or monocyte. In embodiments, the myeloid cell is a cell derived from, or derivable from a monocyte. In embodiments, the myeloid cell is a macrophage. In embodiments, the myeloid cell is a dendritic cell.

In embodiments, the immune cell is an NK cell. In embodiments, the NK cell is a human cell. In embodiments, the NK cell is derived from somatic cell of a subject. In embodiments, the NK cell is derived from allogeneic or autologous cells. In embodiments, the NK cell is derived from an induced pluripotent stem (iPS) cell. In embodiments, the iPS is derived from reprogramming a somatic cell to an iPS cell, the reprogramming comprising contacting the iPS cell with a ribonucleic acid (RNA) encoding one or more reprogramming factors, optionally selected from Oct4, Sox2, cMyc, and Klf4. In embodiments, the iPS cell is derived from allogeneic or autologous cells. In embodiments, the NK cell expresses one or more of CD56 and CD16.

In embodiments, the NK cell expresses CD16a, which optionally binds an antibody/antigen complex on a tumor cell and/or wherein the CD16a is optionally a high affinity variant, optionally homozygous or heterozygous for F158V (See, FIG. 12A and FIG. 12B).

In embodiments, the NK cell does not express CD3 In embodiments, the NK cell is CD56^{bright} CD16^{dim/-}. In embodiments, the NK cell is CD56^{dim} CD16+. In embodiments, the NK cell is a NK^{tolerant} cell, optionally comprising CD56^{brigt} NK cells or CD27-CD11b-NK cells. In embodiments, the NK cell is a NK^{cytotoxic}, optionally comprising CD56^{dim} NK cells or CD11b+ CD27-NK cells. In embodiments, the NK cell is a NW^{regulatoy}, optionally comprising CD56^{bright} NK cells or CD27+NK cells. In embodiments, the NK cell is a natural killer T (NKT) cell.

In embodiments, the NK cell secretes one or more cytokines selected from interferon-gamma (IFN-g), tumor necrosis factor-alpha (TNF-α), tumor necrosis factor-beta (TNF-b), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b).

In embodiments, the present immune cell has reduced or eliminated immune cell fratricide, e.g., NK-cell fratricide. For instance, in embodiments, the present engineered NK cells surprisingly do not engage in NK cytotoxicity and therefore are able to survive despite disruptions, e.g., in beta-2-microglobulin (B2M).

In embodiments, the present immune cell is capable of self-activating. In embodiments, the present immune cell is capable of activating without the need for extracellular signals (e.g., cytokines), including signals that may be provided exogenously. In embodiments, the present immune cell does not require ex vivo stimulation for activity. In embodiments, the present immune cell is capable of self-activating in the absence of an interleukin, optionally selected from IL-2 and IL-15.

In embodiments, the present immune cell is capable of inducing tumor cell cytotoxicity. In embodiments, the present immune cell is capable of inducing tumor cell cytotoxicity in the absence of an interleukin, optionally selected from IL-2 and IL-15. Assays for assessing tumor cell cytotoxicity include in vivo anti-cancer response evaluation, as well as microscopic evaluation, e.g., a calcein acetoxymethyl (AM) staining-based microscopic method (See EXAMPLES and Chava et al. J Vis Exp. 2020 Feb. 22; (156): 10.3791/60714, the entire contents of which are incorporated by reference). Further, a colorimetric lactic dehydrogenase (LDH) measurement-based NK cell-mediated cytotoxicity assay may be employed (see Chava et al. J Vis Exp. 2020 Feb. 22; (156): 10.3791/60714, the entire contents of which are incorporated by reference).

Chimeric Antigen Receptor (CAR)-Bearing Immune Cells

In embodiments, the present immune cells (e.g., cells that are gene-edited and reprogrammed into an immune cell) are engineered with chimeric antigen receptors (CARs), e.g., the present immune cells are CAR-NK cells or CAR-T.

In embodiments, the immune cell, optionally NK cell or T cell, is genetically modified to express a recombinant chimeric antigen receptor (CAR) comprising an intracellular signaling domain, a transmembrane domain, and an extracellular domain comprising an antigen binding region. In embodiments, the intracellular signaling domain comprises at least one immunereceptor tyrosine based activation motif (ITAM)-containing domain.

In embodiments, the intracellular signaling domain is from one of CD3-zeta, CD28, CD27, CD134 (OX40), and CD137 (4-1BB).

In embodiments, the transmembrane domain is from one of CD28 or a CD8.

In embodiments, the antigen binding region binds one antigen. In embodiments, the binding region binds two antigens.

In embodiments, the extracellular domain comprising an antigen binding region comprises: (a) a natural ligand or receptor, or fragment thereof, or (b) an immunoglobulin domain, optionally a single-chain variable fragment (scFv). In embodiments, the extracellular domain comprising an antigen binding region comprises two of (a) a natural ligand or receptor, or fragment thereof, or (b) an immunoglobulin domain, optionally a single-chain variable fragment (scFv). In embodiments, the extracellular domain comprising an antigen binding region comprises one of each of: (a) a natural ligand or receptor, or fragment thereof, and (b) an immunoglobulin domain, optionally a single-chain variable fragment (scFv).

In embodiments, the antigen binding region binds a tumor antigen.

In embodiments, the antigen binding region comprises one or more of (i) CD94/NKG2a, which optionally binds HLA-E on a tumor cell; (ii) CD96, which optionally binds CD155 on a tumor cell; (iii) TIGIT, which optionally binds CD155 or CD112 on a tumor cell; (iv) DNAM-1, which optionally binds CD155 or CD112 on a tumor cell; (v) KIR, which optionally binds HLA class I on a tumor cell; (vi) NKG2D, which optionally binds NKG2D-L on a tumor cell; (vii) CD16 (e.g., CD16a or CD16b), which optionally binds an antibody/antigen complex on a tumor cell and/or wherein the CD16a is optionally a high affinity variant, optionally homozygous or heterozygous for F158V; (viii) NKp30, which optionally binds B7-H6 on a tumor cell; (ix) NKp44; and (x) NKp46.

In embodiments, the antigen binding region comprises an immunoglobulin domain, optionally an scFv directed against HLA-E, CD155, CD112 HLA class I, NKG2D-L, or B7-H6, as well as any variant thereof.

In embodiments, the antigen binding region binds an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, as well as any variant thereof. In embodiments, an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, as well as any variant thereof can be used as a single-target CAR, dual-target CAR, mAb, or any combination of any of those

In embodiments, the antigen binding region binds two antigen, the antigens being: (a) an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2, as well as any variant thereof and (b) an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2, as well as any variant thereof.

In embodiments, the antigen binding region binds two antigen, the antigens being: (a) an antigen selected from CD16, CD64, CD78, CD96, CLL1, CD116, CD117, CD71, CD45, CD71, CD123 and CD138, a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvlll), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-1, IGF-I I, IGFI receptor, mesothelin, a major histocompatibility complex (MHC) molecule presenting a tumor-specific peptide epitope, 5T4, ROR1, Nkp30, N KG2D, tumor stromal antigens, the extra domain A (EDA) and extra domain B (EDB) of fibronectin and the A1 domain of tenascin-C(TnC A1) and fibroblast associated protein (FAP); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF 17), multiple myeloma or lymphoblastic leukemia antigen, such as one selected from TNFRSF17, SLAMF7, GPRC5D, FKBP11, KAMP3, ITGA8, and FCRL5, a virus-specific surface antigen such as an HIV-specific antigen (such as HIV gpl20); an EBV-specific antigen, a CMV-specific antigen, a HPV-specific antigen, a Lasse Virus-specific antigen, an Influenza Virus-specific antigen, as well as any variant thereof and (b) an antigen selected from CD16, CD64, CD78, CD96, CLL1, CD116, CD117, CD71, CD45, CD71, CD123 and CD138, a tumor-associated surface antigen, such as ErbB2 (HER2/neu), carcinoembryonic antigen (CEA), epithelial cell adhesion molecule (EpCAM), epidermal growth factor receptor (EGFR), EGFR variant I II (EGFRvl 11), CD19, CD20, CD30, CD40, disialoganglioside GD2, ductal-epithelial mucine, gp36, TAG-72, glycosphingolipids, glioma-associated antigen, 0-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-1, IGF-I I, IGFI receptor, mesothelin, a major histocompatibility complex (MHC) molecule presenting a tumor-specific peptide epitope, 5T4, ROR1, Nkp30, N KG2D, tumor stromal antigens, the extra domain A (EDA) and extra domain B (EDB) of fibronectin and the A1 domain of tenascin-C(TnC A1) and fibroblast associated protein (FAP); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF 17), multiple myeloma or lymphoblastic leukemia antigen, such as one selected from TNFRSF17, SLAMF7, GPRC5D, FKBP11, KAMP3, ITGA8, and FCRL5, a virus-specific surface antigen such as an HIV-specific antigen (such as HIV gpl20); an EBV-specific antigen, a CMV-specific antigen, a HPV-specific antigen, a Lasse Virus-specific antigen, an Influenza Virus-specific antigen, as well as any variant thereof.

In embodiments, the extracellular domain of the recombinant CAR comprises the extracellular domain of an NK cell activating receptor or a scFv.

In embodiments, the NK cell comprises a gene-edit in one or more of IL-7, CCL17, CCR4, IL-6, IL-6R, IL-12, IL-15, NKG2A, NKG2D, KIR, TRAIL, TRAC, PD1, and HPK1.

In embodiments, the gene-edit in one or more of IL-7, CCL17, CCR4, IL-6, IL-6R, IL-12, IL-15, NKG2A, NKG2D, KIR, TRAIL, TRAC, PD1, and HPK1 is caused by contacting the cell with RNA encoding one or more gene-editing proteins. In embodiments, the gene-edit of causes a reduction or elimination of expression and/or activity of IL-6, NKG2A, NKG2D, KIR, TRAC, PD1, and/or HPK1. In embodiments, the gene-edit causes an increase of expression and/or activity of IL-7, CCL17, CCR4, IL-6R, IL-12, IL-15, and/or TRAIL.

In embodiments, the immune cell, e.g., a T cell, NK cell, or macrophage, further comprises one or more recombinant genes capable of encoding a suicide gene product. In embodiments, the suicide gene product comprises a protein selected from the group consisting of thymidine kinase and an apoptotic signaling protein.

Any immune cell disclosed herein (i.e., comprising a gene edit (e.g., in B2M), expressing a high affinity CD16a receptor, and/or expressing a fusion protein comprising B2M polypeptide and an HLA polypeptide) can be further genetically engineered to express a CAR.

RNA Modifications

In embodiments, the present disclosure relates to RNA-based modifications, e.g., reprogramming and/or gene-editing. In some embodiments, a RNA molecule encodes a gene-editing protein. In some embodiments, a RNA molecule encodes a reprogramming factor.

In embodiments, the RNA is mRNA. In embodiments, the RNA is modified mRNA. In embodiments, the modified mRNA comprises one or more non-canonical nucleotides.

In some embodiments, non-canonical nucleotides are incorporated into RNA to increase the efficiency with which the RNA can be translated into protein, and can decrease the toxicity of the RNA. In embodiments, the RNA molecule comprises one or more non-canonical nucleotides. In some embodiments, the nucleic acid comprises one or more non-canonical nucleotide members of the 5-methylcytidine de-methylation pathway. In some embodiments, the nucleic acid comprises at least one of 5-methylcytidine, 5-hydroxymethylcytidine, 5-formylcytidine, and 5-carboxycytidine or a derivative thereof. In some embodiments, the nucleic acid comprises at least one of: pseudouridine, 5-methylpseudouridine, 5-methyluridine, 5-methylcytidine, 5-hydroxymethylcytidine, N4-methylcytidine, N4-acetylcytidine, and 7-deazaguanosine or a derivative thereof.

In embodiments, the non-canonical nucleotides have one or more substitutions at positions selected from the 2C, 4C, and 5C positions for a pyrimidine, or selected from the 6C, 7N and 8C positions for a purine.

In embodiments, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In some embodiments, the one or more non-canonical nucleotides are selected from: 5-methyluridine and 5-methylcytidine, 5-methyluridine and 7-deazaguanosine, 5-methylcytidine and 7-deazaguanosine, 5-methyluridine, 5-methylcytidine, and 7-deazaguanosine, and 5-methyluridine, 5-hydroxymethylcytidine, and 7-deazaguanosine. In some embodiments, the RNA molecule comprises at least two of: 5-methyluridine, 5-methylcytidine, 5-hydroxymethylcytidine, and 7-deazaguanosine or one or more derivatives thereof. In some embodiments, the RNA molecule comprises at least three of 5-methyluridine, 5-methylcytidine, 5-hydroxymethylcytidine, and 7-deazaguanosine or one or more derivatives thereof. In embodiments, the mRNA comprises one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In embodiments, at least 50%, at least 60%, at least 70%, at least 80%, at least 90%, or 100% of the non-canonical nucleotides comprises one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, the RNA molecule comprises at least one of: one or more uridine residues, one or more cytidine residues, and one or more guanosine residues, and comprising one or more non-canonical nucleotides. In one embodiment, between about 20% and about 80% of the uridine residues are 5-methyluridine residues. In another embodiment, between about 30% and about 50% of the uridine residues are 5-methyluridine residues. In a further embodiment, about 40% of the uridine residues are 5-methyluridine residues. In one embodiment, between about 60% and about 80% of the cytidine residues are 5-methylcytidine residues. In another embodiment, between about 80% and about 100% of the cytidine residues are 5-methylcytidine residues. In a further embodiment, about 100% of the cytidine residues are 5-methylcytidine residues. In a still further embodiment, between about 20% and about 100% of the cytidine residues are 5-hydroxymethylcytidine residues. In one embodiment, between about 20% and about 80% of the guanosine residues are 7-deazaguanosine residues. In another embodiment, between about 40% and about 60% of the guanosine residues are 7-deazaguanosine residues. In a further embodiment, about 50% of the guanosine residues are 7-deazaguanosine residues. In one embodiment, between about 20% and about 80% or between about 30% and about 60% or about 40% of the cytidine residues are N4-methylcytidine and/or N4-acetylcytidine residues. In another embodiment, each cytidine residue is a 5-methylcytidine residue. In a further embodiment, about 100% of the cytidine residues are 5-methylcytidine residues and/or 5-hydroxymethylcytidine residues and/or N4-methylcytidine residues and/or N4-acetylcytidine residues and/or one or more derivatives thereof. In a still further embodiment, about 40% of the uridine residues are 5-methyluridine residues, between about 20% and about 100% of the cytidine residues are N4-methylcytidine and/or N4-acetylcytidine residues, and about 50% of the guanosine residues are 7-deazaguanosine residues. In one embodiment, about 40% of the uridine residues are 5-methyluridine residues and about 100% of the cytidine residues are 5-methylcytidine residues. In another embodiment, about 40% of the uridine residues are 5-methyluridine residues and about 50% of the guanosine residues are 7-deazaguanosine residues. In a further embodiment, about 100% of the cytidine residues are 5-methylcytidine residues and about 50% of the guanosine residues are 7-deazaguanosine residues. In one embodiment, about 40% of the uridine residues are 5-methyluridine residues, about 100% of the cytidine residues are 5-methylcytidine residues, and about 50% of the guanosine residues are 7-deazaguanosine residues. In another embodiment, about 40% of the uridine residues are 5-methyluridine residues, between about 20% and about 100% of the cytidine residues are 5-hydroxymethylcytidine residues, and about 50% of the guanosine residues are 7-deazaguanosine residues. In some embodiments, less than 100% of the cytidine residues are 5-methylcytidine residues. In other embodiments, less than 100% of the cytidine residues are 5-hydroxymethylcytidine residues. In one embodiment, each uridine residue in the RNA molecule is a pseudouridine residue or a 5-methylpseudouridine residue. In another embodiment, about 100% of the uridine residues are pseudouridine residues and/or 5-methylpseudouridine residues. In a further embodiment, about 100% of the uridine residues are pseudouridine residues and/or 5-methylpseudouridine residues, about 100% of the cytidine residues are 5-methylcytidine residues, and about 50% of the guanosine residues are 7-deazaguanosine residues.

Other non-canonical nucleotides that can be used in place of or in combination with 5-methyluridine include but are not limited to: pseudouridine and 5-methylpseudouridine (a.k.a. “1-methylpseudouridine”, a.k.a. “N1-methylpseudouridine”) or one or more derivatives thereof. Other non-canonical nucleotides that can be used in place of or in combination with 5-methylcytidine and/or 5-hydroxymethylcytidine include, but are not limited to: pseudoisocytidine, 5-methylpseudoisocytidine, 5-hydroxymethylcytidine, 5-formylcytidine, 5-carboxycytidine, N4-methylcytidine, N4-acetylcytidine or one or more derivatives thereof. In certain embodiments, for example, when performing only a single transfection or when the cells being transfected are not particularly sensitive to transfection-associated toxicity or innate-immune signaling, the fractions of non-canonical nucleotides can be reduced. Reducing the fraction of non-canonical nucleotides can be beneficial, in part, because reducing the fraction of non-canonical nucleotides can reduce the cost of the nucleic acid. In certain situations, for example, when minimal immunogenicity of the nucleic acid is desired, the fractions of non-canonical nucleotides can be increased.

In embodiments, the RNA molecule comprises a 5′ cap structure. In embodiments, the RNA molecule comprises a 5′-UTR comprising a Kozak consensus sequence. In embodiments, the RNA molecule comprises a 5′-UTR comprising a sequence that increases RNA stability in vivo. In embodiments, the RNA molecule comprises a 3′-UTR comprising a sequence that increases RNA stability in vivo. In embodiments, the 5′-UTR comprises an alpha-globin or beta-globin 5′-UTR sequence. In embodiments, the 3′-UTR comprises an alpha-globin or beta-globin 3′-UTR sequence. In embodiments, the RNA molecule comprises a 3′ poly(A) tail.

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is between about 20nt and about 250nt or between about 120nt and about 150nt long. In a further embodiment, the 3′-poly(A) tail is about 20nt, or about 30nt, or about 40nt, or about 50nt, or about 60nt, or about 70nt, or about 80nt, or about 90nt, or about 100nt, or about 110nt, or about 120nt, or about 130nt, or about 140nt, or about 150nt, or about 160nt, or about 170nt, or about 180nt, or about 190nt, or about 200nt, or about 210nt, or about 220nt, or about 230nt, or about 240nt, or about 250nt long.

In some embodiments, the RNA comprises a tail composed of a plurality of adenines with one or more guanines.

In embodiments, the RNA comprises (a) a sequence encoding a protein, and (b) a tail region comprising deoxyadenosine nucleotides and one or more other nucleotides.

In embodiments, the one or more other nucleotides comprises deoxyguanosine residues. In embodiments, the tail region comprises about 1%, about 2%, about 5%, about 10%, about 15%, about 20%, about 25%, about 30%, about 35%, about 40%, about 45%, or about 50% deoxyguanosine residues. In embodiments, the tail region comprises more than 50% deoxyguanosine residues.

In embodiments, the one or more other nucleotides comprises deoxycytidine residues. In embodiments, the tail region comprises about 1%, about 2%, about 5%, about 10%, about 15%, about 20%, about 25%, about 30%, about 35%, about 40%, about 45%, or about 50% deoxycytidine residues. In embodiments, the tail region comprises more than 50% deoxycytidine residues.

In embodiments, the one or more other nucleotides comprises deoxythymidine residues. In embodiments, the tail region comprises about 1%, about 2%, about 5%, about 10%, about 15%, about 20%, about 25%, about 30%, about 35%, about 40%, about 45%, or about 50% deoxythymidine residues. In embodiments, the tail region comprises more than 50% deoxythymidine residues.

In embodiments, the one or more other nucleotides comprise deoxyguanosine residues and deoxycytidine residues. In embodiments, the tail region comprises about 99%, about 98%, about 95%, about 90%, about 85%, about 80%, about 75%, about 70%, about 65%, about 60%, about 55%, or about 50% deoxyadenosine residues. In embodiments, the tail region comprises fewer than 50% deoxyadenosine residues.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues. In embodiments, the tail region comprises more than 50% guanosine residues.

In embodiments, the one or more other nucleotides comprises cytidine residues. In embodiments, the tail region comprises about 1%, about 2%, about 5%, about 10%, about 15%, about 20%, about 25%, about 30%, about 35%, about 40%, about 45%, or about 50% cytidine residues. In embodiments, the tail region comprises more than 50% cytidine residues.

In embodiments, the one or more other nucleotides comprises uridine residues. In embodiments, the tail region comprises about 1%, about 2%, about 5%, about 10%, about 15%, about 20%, about 25%, about 30%, about 35%, about 40%, about 45%, or about 50% uridine residues. In embodiments, the tail region comprises more than 50% uridine residues.

In embodiments, the one or more other nucleotides comprise guanosine residues and cytidine residues. In embodiments, the tail region comprises about 99%, about 98%, about 95%, about 90%, about 85%, about 80%, about 75%, about 70%, about 65%, about 60%, about 55%, or about 50% adenosine residues.

In embodiments, the tail region comprises fewer than 50% adenosine residues.

In embodiments, the tail is(A)₁₅₀. In embodiments, the tail is (A₃₉G)₃(A)₃₀. In embodiments, the tail is (A₁₉G)₇(A)₁₀. In embodiments, the tail is (A₉G)₁₅.

In embodiments, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In embodiments, the length of the tail region is greater than 320 nucleotides.

In embodiments, the RNA comprises a 5′ cap structure. In embodiments, the RNA 5′-UTR comprises a Kozak consensus sequence. In embodiments, the RNA 5′-UTR comprises a sequence that increases RNA stability in vivo, and the 5′-UTR may comprise an alpha-globin or beta-globin 5′-UTR.

In embodiments, the RNA 3′-UTR comprises a sequence that increases RNA stability in vivo, and the 3′-UTR may comprise an alpha-globin or beta-globin 3′-UTR. In embodiments, the RNA comprises a 3′ poly(A) tail. In embodiments, the RNA 3′ poly(A) tail is from about 20 nucleotides to about 250 nucleotides in length.

In embodiments, the RNA is from about 200 nucleotides to about 5000 nucleotides in length.

In embodiments, the RNA is prepared by in vitro transcription. In embodiments, the RNA is synthetic.

Gene-Editing Proteins

In embodiments, the present disclosure relates to gene editing to provide a genetically engineered disruption in a gene, e.g., beta-2-microglobulin (B2M). In embodiments, the gene-editing is undertaken using a RNA molecule encoding a gene-editing protein.

In embodiments, the gene-editing protein is selected from a nuclease, a transcription activator-like effector nuclease (TALEN), RiboSlice, a zinc-finger nuclease, a meganuclease, a nickase, a clustered regularly interspaced short palindromic repeat (CRISPR)-associated protein or a natural or engineered variant, family-member, orthologue, fragment or fusion construct thereof.

In embodiments, the gene-editing protein comprises: (i) a DNA-binding domain comprising a plurality of repeat sequences and (ii) the nuclease domain comprising a catalytic domain of a nuclease. In embodiments, the at least one of the repeat sequences comprises the amino acid sequence: LTPvQVVAIAwxyza (SEQ ID NO: 3) and is optionally between 36 and 39 amino acids long, where:
v is Q, D or E,
w is S or N,
x is I, H, N, or I,
y is D, A, I, N, H, K, S, G, or null,
z is GGRPALE (SEQ ID NO: 4), GGKQALE (SEQ ID NO: 5), GGKQALETVQRLLPVLCQDHG (SEQ ID NO: 6), GGKQALETVQRLLPVLCQAHG (SEQ ID NO: 7), GKQALETVQRLLPVLCQDHG (SEQ ID NO: 8), GKQALETVQRLLPVLCQAHG (SEQ ID NO: 9), GGKQALETVQRLLPVLCQD (SEQ ID NO: 10) or GGKQALETVQRLLPVLCQA (SEQ ID NO: 11), and
α is four consecutive amino acids.

In embodiments, α comprises at least one glycine (G) residue. In embodiments, a comprises at least one histidine (H) residue. In embodiments, a comprises at least one histidine (H) residue at any one of positions 33, 34, or 35. In embodiments, a comprises at least one aspartic acid (D) residue. In embodiments, α comprises at least one, or two, or three of a glycine (G) residue, a histidine (H) residue, and an aspartic acid (D) residue.

In embodiments, α comprises one or more hydrophilic residues, optionally selected from: a polar and positively charged hydrophilic amino acid, optionally selected from arginine (R) and lysine (K); a polar and neutral of charge hydrophilic amino acid, optionally selected from asparagine (N), glutamine (Q), serine (S), threonine (T), proline (P), and cysteine (C); a polar and negatively charged hydrophilic amino acid, optionally selected from aspartate (D) and glutamate (E), and an aromatic, polar and positively charged hydrophilic amino acid, optionally selected from histidine (H).

In some embodiments, α comprises one or more polar and positively charged hydrophilic amino acids selected from arginine (R) and lysine (K). In some embodiments, α comprises one or more polar and neutral of charge hydrophilic amino acids selected from asparagine (N), glutamine (Q), serine (S), threonine (T), proline (P), and cysteine (C). In some embodiments, α comprises one or more polar and negatively charged hydrophilic amino acids selected from aspartate (D) and glutamate (E). In some embodiments, α comprises one or more aromatic, polar and positively charged hydrophilic amino acids selected from histidine (H).

In embodiments, α comprises one or more hydrophobic residues, optionally selected from: a hydrophobic, aliphatic amino acid, optionally selected from glycine (G), alanine (A), leucine (L), isoleucine (I), methionine (M), and valine (V), and a hydrophobic, aromatic amino acid, optionally selected from phenylalanine (F), tryptophan (W), and tyrosine (Y). In some embodiments, α comprises one or more hydrophobic, aliphatic amino acids selected from glycine (G), alanine (A), leucine (L), isoleucine (I), methionine (M), and valine (V). In some embodiments, α comprises one or more aromatic amino acids selected from phenylalanine (F), tryptophan (W), and tyrosine (Y). In embodiments, the DNA-binding domain comprises about 15, or about, 16, or about 17, or about 18, or about 18.5 repeat sequences.

In embodiments, a is selected from GHGG (SEQ ID NO: 12), HGSG (SEQ ID NO: 13), HGGG (SEQ ID NO: 14), from GGHD (SEQ ID NO: 15), GAHD (SEQ ID NO: 16), AHDG (SEQ ID NO: 17), PHDG (SEQ ID NO: 18), GPHD (SEQ ID NO: 19), GHGP (SEQ ID NO: 20), PHGG (SEQ ID NO: 21), PHGP (SEQ ID NO: 22), AHGA (SEQ ID NO: 23), LHGA (SEQ ID NO: 24), VHGA (SEQ ID NO: 25), IVHG (SEQ ID NO: 26), IHGM (SEQ ID NO: 27), RHGD (SEQ ID NO: 28), RDHG (SEQ ID NO: 29), RHGE (SEQ ID NO: 30), HRGE (SEQ ID NO: 31), RHGD (SEQ ID NO: 32), HRGD (SEQ ID NO: 33), GPYE (SEQ ID NO: 34), NHGG (SEQ ID NO: 35), THGG (SEQ ID NO: 36), GTHG (SEQ ID NO: 37), GSGS (SEQ ID NO: 38), GSGG (SEQ ID NO: 39), GGGG (SEQ ID NO: 40), GRGG (SEQ ID NO: 41), and GKGG (SEQ ID NO: 42).

In embodiments, the gene-editing protein comprises a repeat variable di-residue (RVD) at residue 12 or 13 which targets the DNA-binding domain to a target DNA molecule.

In embodiments, the RVD recognizes one base pair in the nucleic acid molecule. In embodiments, the RVD recognizes a C residue in the nucleic acid molecule and is selected from HD, N(null), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H(null), and IG.

In embodiments, the RVD recognizing a C residue in the nucleic acid molecule is HD. In some embodiments, the RVD recognizing a C residue in the nucleic acid molecule is N(null). In some embodiments, the RVD recognizing a C residue in the nucleic acid molecule is HA. In some embodiments, the RVD recognizing a C residue in the nucleic acid molecule is ND. In some embodiments, the RVD recognizing a C residue in the nucleic acid molecule is HI. In some embodiments, the RVD recognizing a G residue in the nucleic acid molecule is NN. In some embodiments, the RVD recognizing a G residue in the nucleic acid molecule is NH. In some embodiments, the RVD recognizing a G residue in the nucleic acid molecule is NK. In some embodiments, the RVD recognizing a G residue in the nucleic acid molecule is HN. In some embodiments, the RVD recognizing a G residue in the nucleic acid molecule is NA. In some embodiments, the RVD recognizing an A residue in the nucleic acid molecule is NI. In some embodiments, the RVD recognizing an A residue in the nucleic acid molecule is NS. In some embodiments, the RVD recognizing a T residue in the nucleic acid molecule is NG. In some embodiments, the RVD recognizing a T residue in the nucleic acid molecule is HG. In some embodiments, the RVD recognizing a T residue in the nucleic acid molecule is H(null). In some embodiments, the RVD recognizing a T residue in the nucleic acid molecule is IG.

In embodiments, the gene-editing protein has a DNA binding domain having at least one repeat of LTPEQVVAIAS*RVD*GGKQALETVQRLLPVLCQAGHGG (SEQ ID NO: 43; the “*RVD*” corresponds to the dinucleotide “xy” of SEQ ID NO:3).

In embodiments, the repeat sequence is 33 or 34 amino acids long. In embodiments, the repeat sequence is 36-39 amino acids long. In some embodiments, the repeat sequence is 36 amino acids long. In some embodiments, the repeat sequence is 37 amino acids long. In some embodiments, the repeat sequence is 38 amino acids long. In some embodiments, the repeat sequence is 39 amino acids long.

In embodiments, the nuclease domain comprises a catalytic domain of a nuclease. In embodiments, the nuclease domain is capable of forming a dimer with another nuclease domain. In embodiments, the nuclease is selected from FokI, StsI, or a hybrid thereof repeat sequences. In embodiments, the catalytic domain is hybrid of the catalytic domains of FokI and StsI comprising the α1, α2, α3, α4, α5, α6, β1, β2, β3, β4, β5, and 36 domains of FokI with at least one of the domains of FokI being substituted in whole or in part with the α1, α2, α3, α4, α5, α6, β1, β2, β3, β4, β5, and β6 domains of StsI and optionally comprising at least one mutation.

In some embodiments, certain fragments of an endonuclease cleavage domain are used, including fragments that are truncated at the N-terminus, fragments that are truncated at the C-terminus, fragments that have internal deletions, and fragments that combine N-terminus, C-terminus, and/or internal deletions, which maintain part or all of the catalytic activity of the full endonuclease cleavage domain. Determining whether a fragment can maintain part or all of the catalytic activity of the full domain can be accomplished by, for example, synthesizing a gene-editing protein that contains the fragment according to the methods of the present invention, inducing cells to express the gene-editing protein according to the methods of the present invention, and measuring the efficiency of gene editing. In some embodiments, a measurement of gene-editing efficiency is used to ascertain whether any specific fragment maintains part or all of the catalytic activity of the full endonuclease cleavage domain. Certain embodiments are therefore directed to a biologically active fragment of an endonuclease cleavage domain. In one embodiment, the endonuclease cleavage domain is selected from: FokI, StsI, StsI-HA, StsI-HA2, StsI-UHA, StsI-UHA2, StsI-HF, and StsI-UHF or a natural or engineered variant or biologically active fragment thereof, or a hybrid or chimera thereof.

In embodiments, the gene-editing protein comprises a linker. In another embodiment, the linker connects a DNA-binding domain to a nuclease domain. In a further embodiment, the linker is between about 1 and about 10 amino acids long. In some embodiments, the linker is about 1, about 2, or about 3, or about 4, or about 5, or about 6, or about 7, or about 8, or about 9, or about 10 amino acids long. In one embodiment, the gene-editing protein is capable of generating a nick or a double-strand break in a target DNA molecule.

In embodiments, the gene-editing protein is any of those described in International Patent Publication No. WO 2014/071219 or U.S. Provisional Application No. 63/023,678, hereby incorporated by reference in their entireties.

Formulations Administration

In some embodiments, the present disclosure relates to compositions described herein in the form of a pharmaceutical composition.

In various embodiments, the present invention pertains to pharmaceutical compositions comprising the immune cell described herein and a pharmaceutically acceptable carrier or excipient. In some embodiments, the present invention pertains to pharmaceutical compositions comprising the present immune cell.

Lipids Cell Contacting Transfection

In embodiments, the present invention relates delivery of the present RNA molecules via a lipid. In embodiments, the present mRNAs encoding a gene-editing protein and/or reprogramming factor are delivered via a lipid.

In embodiments, the lipid is a compound of Formula (I)

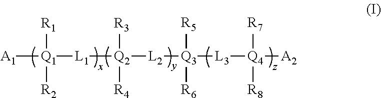

wherein: Q₁, Q₂, Q₃, and Q₄ are independently an atom or group capable of adopting a positive charge;
A₁ and A₂ are independently null, H, or optionally substituted C₁-C₆ alkyl;
L₁, L₂, and L₃ are independently null, a bond, (C₁-C₂₀)alkanediyl, (halo)(C₁-C₂₀)alkanediyl, (hydroxy)(C₁-C₂₀)alkanediyl, (alkoxy)(C₁-C₂₀)alkanediyl, arylene, heteroarylene, cycloalkanediyl, heterocycle-diyl, or any combination of the aforementioned optionally linked by one or more of an ether, an ester, an anhydride, an amide, a carbamate, a secondary amine, a tertiary amine, a quaternary ammonium, a thioether, a urea, a carbonyl, or an imine;
R₁, R₂, R₃, R₄, R₅, R₆, R₇, and R₈ are independently null, H, (C₁-C₆₀)alkyl, (halo)(C₁-C₆₀)alkyl, (hydroxy)(C₁-C₆₀)alkyl, (alkoxy)(C₁-C₆₀)alkyl, (C₂-C₆₀)alkenyl, (halo)(C₂-C₆₀)alkenyl, (hydroxy)(C₂-C₆₀)alkenyl, (alkoxy)(C₂-C₆₀)alkenyl, (C₂-C₆₀)alkynyl, (halo)(C₂-C₆₀)alkynyl, (hydroxy)(C₂-C₆₀)alkynyl, (alkoxy)(C₂-C₆₀)alkynyl, wherein at least one of R₁, R₂, R3, R4, R5, R6, R7, and R8 comprises at least two unsaturated bonds; and x, y, and z are independently 0, 1, 2, 3, 4, 5, 6, 7, 8, 9, 10, 11, 12, 13, 14, or 15.

In embodiments, the lipid is a compound of Formula (II):

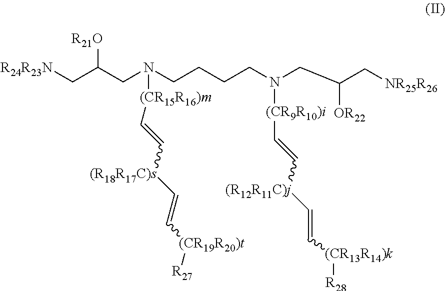

wherein: R9, R10, R11, R12, R13, R14, R15, R16, R17, R18, R19, R20, R21, R22, R23, R24, R25, R26, R27, and R28 are independently H, halo, OH, (C1-C6)alkyl, (halo)(C1-C6)alkyl, (hydroxy)(C1-C6)alkyl, (alkoxy)(C1-C₆)alkyl, aryl, heteroaryl, cycloalkyl, or heterocyclo; and
i, j, k, m, s, and t are independently 0, 1, 2, 3, 4, 5, 6, 7, 8, 9, 10, 11, 12, 13, 14, or 15.

In embodiments, the lipid is a compound of Formula (III):

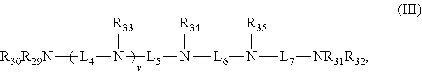

wherein L₄, L₅, L₆, and L₇ are independently a bond, (C₁-C₂₀)alkanediyl, (halo)(C₁-C₂₀)alkanediyl, (hydroxy)(C₁-C₂₀)alkanediyl, (alkoxy)(C₁-C₂₀)alkanediyl, arylene, heteroarylene, cycloalkanediyl, heterocycle-diyl, —(CH₂)ᵥ₁—C(O)—, —((CH₂)ᵥ₁—O)ᵥ₂—, or —((CH₂)ᵥ₁—C(O)—O)ᵥ₂—; R₂₉, R₃₀, R₃₁, R₃₂, R₃₃, R₃₄, and R₃₅ are independently H, (C₁-C₆₀)alkyl, (halo)(C₁-C₆₀)alkyl, (hydroxy)(C₁-C₆₀)alkyl, (alkoxy)(C₁-C₆₀)alkyl, (C₂-C₆₀)alkenyl, (halo)(C₂-C₆₀)alkenyl, (hydroxy)(C₂-C₆₀)alkenyl, (alkoxy)(C₂-C₆₀)alkenyl, (C₂-C₆₀)alkynyl, (halo)(C₂-C₆₀)alkynyl, (hydroxy)(C₂-C₆₀)alkynyl, (alkoxy)(C₂-C₆₀)alkynyl, wherein at least one of R₂₉, R₃₀, R₃₁, R₃₂, R₃₃, R₃₄, and R₃₅ comprises at least two unsaturated bonds;
v, v₁ and v₂ are independently 0, 1, 2, 3, 4, 5, 6, 7, 8, 9, 10, 11, 12, 13, 14, or 15.

In embodiments, the lipid is a compound of Formula (IV):

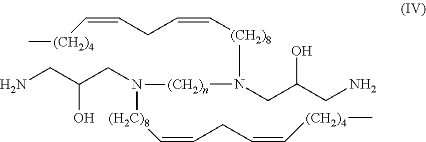

wherein n is 1, 2, 3, 4, 5, 6, 7, 8, 9, 10, 11, 12, 13, 14, or 15.

In embodiments, the lipid is a compound of Formula (V):

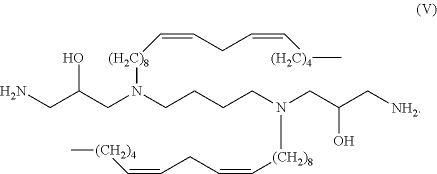

In embodiments, the lipid is a compound of Formula (VI):

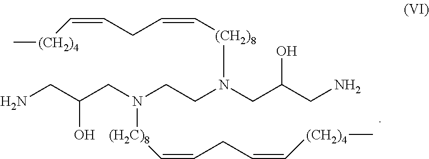

In embodiments, the lipid is a compound of Formula (VII):

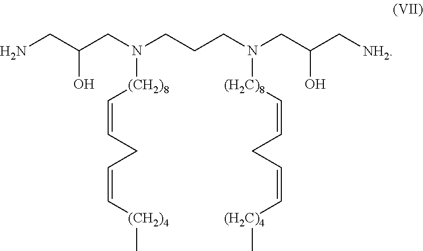

In embodiments, the lipid is a compound of Formula (VIII):

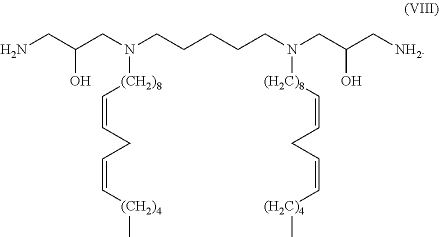

In embodiments, the lipid is a compound of Formula (IX):

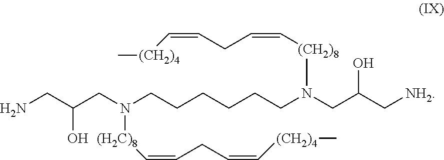

In embodiments, the lipid is a compound of Formula (X):

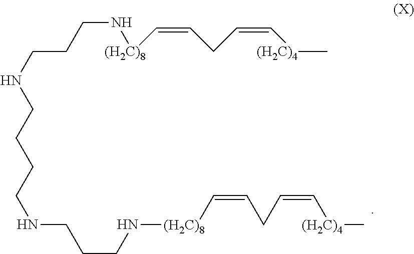

In embodiments, the lipid is a compound of Formula (XI):

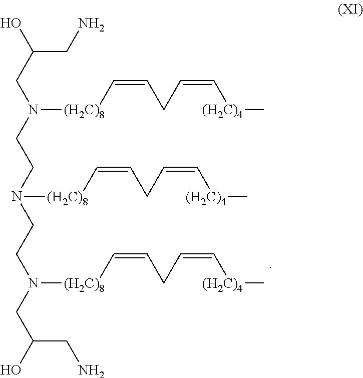

In embodiments, the lipid is a compound of Formula (XII):

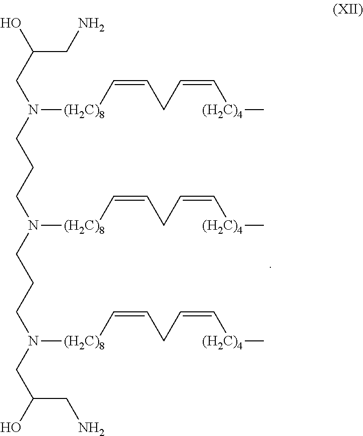

In embodiments, the lipid is a compound of Formula (XIII):

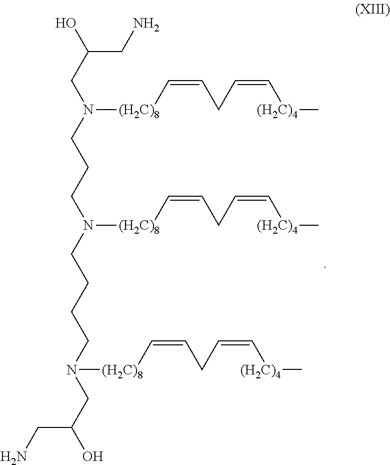

In embodiments, the lipid is a compound of Formula (XIV):

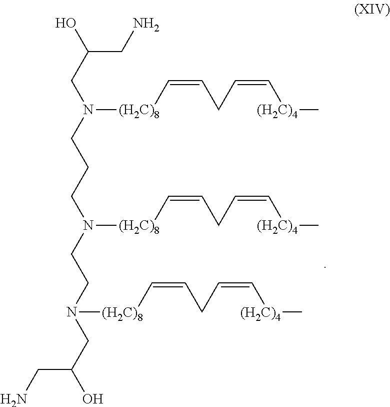

In embodiments, the lipid is a compound of Formula (XV):

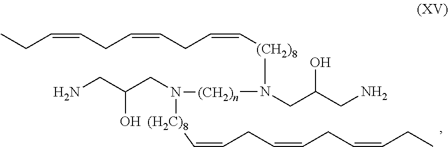

wherein n is 1, 2, 3, 4, 5, 6, 7, 8, 9, 10, 11, 12, 13, 14, or 15.

In embodiments, the lipid is a compound of Formula (XVI):

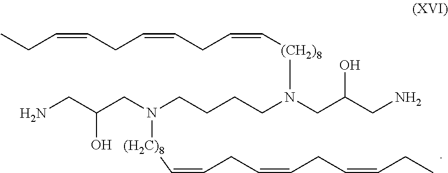

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle.

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle which does not require an additional or helper lipid. In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle that further comprises a neutral lipid (e.g., dioleoylphosphatidylethanolamine (DOPE), 1,2-Dioleoyl-sn-glycero-3-phosphocholine (DOPC), or cholesterol) and/or a further cationic lipid (e.g., N-[1-(2,3-dioleoyloxy)propyl]-N,N,N-trimethylammonium chloride (DOTMA), 1,2-bis(oleoyloxy)-3-3-(trimethylammonium) propane (DOTAP), or 1,2-dioleoyl-3-dimethylammonium-propane (DODAP)).

In embodiments, the lipid is any of those described in International Patent Publication No. WO 2021/003462, hereby incorporated by reference in its entirety.

In embodiments, the lipid is any of those of Table A.

TABLE A
Illustrative Biocompatible Lipids and Polymers

3β-[N-(N′,N′-dimethylaminoethane)-carbamoyl]cholesterol
(DC-Cholesterol)
1,2-dioleoyl-3-trimethylammonium-propane (DOTAP/18:1 TAP)
N-(4-carboxybenzyl)-N,N-dimethyl-2,3-bis(oleoyloxy)propan-1-
aminium (DOBAQ)
1,2-dimyristoyl-3-trimethylammonium-propane (14:0 TAP)
1,2-dipalmitoyl-3-trimethylammonium-propane (16:0 TAP)
1,2-stearoyl-3-trimethylammonium-propane (18:0 TAP)
1,2-dioleoyl-3-dimethylammonium-propane (DODAP/18:1 DAP)
1,2-dimyristoyl-3-dimethylammonium-propane (14:0 DAP)
1,2-dipalmitoyl-3-dimethylammonium-propane (16:0 DAP)
1,2-distearoyl-3-dimethylammonium-propane (18:0 DAP)
dimethyldioctadecylammonium (18:0 DDAB)
1,2-dilauroyl-sn-glycero-3-ethylphosphocholine (12:0 EthylPC)
1,2-dimyristoyl-sn-glycero-3-ethylphosphocholine (14:0 EthylPC)
1,2-dimyristoleoyl-sn-glycero-3-ethylphosphocholine (14:1 EthylPC)
1,2-dipalmitoyl-sn-glycero-3-ethylphosphocholine (16:0 EthylPC)
1,2-distearoyl-sn-glycero-3-ethylphosphocholine (18:0 EthylPC)
1,2-dioleoyl-sn-glycero-3-ethylphosphocholine (18:1 EthylPC)
1-palmitoyl-2-oleoyl-sn-glycero-3-ethylphosphocholine (16:1-18:1
EthylPC)
1,2-di-O-octadecenyl-3-trimethylammonium propane (DOTMA)
N1-[2-((1S)-1-[(3-aminopropyl)amino]-4-[di(3-amino-
propyl)amino]butylcarboxamido)ethyl]-3,4-di[oleyloxy]-benzamide
(MVL5)
2,3-dioleyloxy-N-[2-spermine carboxamide]ethyl-N,N-dimethyl-1-
propanammonium trifluoroacetate (DOSPA)
1,3-di-oleoyloxy-2-(6-carboxy-spermyl)-propylamid (DOSPER)
N-[1-(2,3-dimyristyloxy)propyl]-N,N-dimethyl-N-(2-
hydroxyethyl)ammonium bromide (DMRIE)
LIPOFECTAMINE, LIPOFECTAMINE 2000, LIPOFECTAMINE
RNAiMAX, LIPOFECTAMINE 3000, LIPOFECTAMINE
MessengerMAX, TransIT mRNA
dioctadecyl amidoglyceryl spermine (DOGS)
dioleoyl phosphatidyl ethanolamine (DOPE)
1,2-dilinoleyloxy-3-dimethylaminopropane (DLinDMA)
1,2-dilinoleyl-4-(2-dimethylaminoethyl)-[1,3]-dioxolane (DLin-
KC2-DMA)
Heptatriaconta-6,9,28,31-tetraen-19-yl 4-(dimethylamino)butanoate
(DLin-MC3-DMA)
N1,N4-dimyristyl-N1,N4-di-(2-hydroxy-3-aminopropyl)-
diaminobutane (DHDMS)
N1,N4-dioleyl-N1,N4-di-(2-hydroxy-3-aminopropyl)-diaminobutane
(DHDOS)
1,2-distearoyl-sn-glycero-3-phosphocholine (18:0 PC DSPC)
1,2-dioleyl-sn-glycero-3-phosphocholine (18:1 PC)
1,2-distearyl-sn-glycero-3-phosphatidyl ethanolamine (DSPE)
1,2-dilinoleyl-3-dimethylammonium-propane (18:2 DAP)
hexadimethrine bromide (Polybrene ™)
DEAE-Dextran
protamine
protamine sulfate
poly-L-lysine
poly-D-lysine
Poly(beta-amino-ester) polymer
polyethyleneimine
block co-polymer comprising one or more of: PEG, PLGA, PPG, PEI,
PLL, PCL,
a PLURONIC

Methods of Making

In aspects, the present disclosure provides a method of making an engineered immune cell, comprising: (a) reprogramming a somatic cell to an iPS cell, the reprogramming comprising contacting the iPS cell with a ribonucleic acid (RNA) encoding one or more reprogramming factors; (b) disrupting a beta-2-microglobulin (B2M) gene in the iPS cell, the disrupting comprising gene-editing the cell by contacting the cell with RNA encoding one or more gene-editing proteins; and (c) differentiating the iPS cell into an immune cell, where the immune cell is selected from a lymphoid cell or a myeloid cell. In some cases the lymphoid cell is a T cell, e.g., a cytotoxic T cell or gamma-delta T cell; an NK cell; or an NK-T cell. In some cases, the myeloid cell is a macrophage, e.g., an M1 macrophage or an M2 macrophage.

In embodiments, the method further comprises disrupting a CIITA gene in the iPS cell, the disrupting comprising gene-editing the cell by contacting the cell with RNA encoding one or more gene-editing proteins.

In embodiments, the immune cell is an NK cell.

In embodiments, the somatic cell is a fibroblast or keratinocyte.

In embodiments, the method provides an increased proliferation rate of iPS cells as compared to the rate of iPS cells without a disruption of the B2M gene.

In embodiments, the method provides an increased proliferation rate of differentiating cells along a lymphoid lineage cells as compared to the rate of iPS cells without a disruption of the B2M gene.

In embodiments, the method provides an increased expansion of differentiating cells along a lymphoid lineage cells as compared to the rate of iPS cells without a disruption of the B2M gene

In embodiments, the differentiating comprises embryoid body-based hematopoietic commitment.

In embodiments, the differentiating comprises enrichment of CD34+ cells. In embodiments, the differentiating comprises differentiating into CD5+/CD7+ common lymphoid progenitors.

In embodiments, the method yields CD56^{dim} CD16+ NK cells.

In embodiments, the RNA is associated with one or more lipid selected from and/or Formulae I-XVI.

Methods of Treatment

In aspects, the present disclosure provides a method of treating cancer, comprising: (a) obtaining an isolated immune cell comprising a genetically engineered disruption in a beta-2-microglobulin (B2M) gene; and (b) administering the isolated immune cell to a subject in need thereof, where the immune cell is selected from a lymphoid cell or a myeloid cell.

In some cases the lymphoid cell is a T cell, e.g., a cytotoxic T cell or gamma-delta T cell; an NK cell; or an NK-T cell.

In some cases, the myeloid cell is a macrophage, e.g., an M1 macrophage or an M2 macrophage.

In embodiments, the immune cell is an NK cell.

In embodiments, the cancer is a blood cancer. In embodiments, the cancer is a solid tumor. In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs' syndrome.

The immune cell of the present disclosure may be administered systemically (e.g., via a vein or artery) or may be introduced into a tumor or in the vicinity of the tumor.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular cases only and is not intended to be limiting.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value. Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

By preventing is meant, at least, avoiding the occurrence of a disease and/or reducing the likelihood of acquiring the disease. By treating is meant, at least, ameliorating or avoiding the effects of a disease, including reducing a sign or symptom of the disease.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

Any aspect or embodiment described herein can be combined with any other aspect or embodiment as disclosed herein.

### EMBODIMENTS

Embodiment 1. A composition comprising an isolated immune cell comprising a genetically engineered disruption in a beta-2-microglobulin (B2M) gene, wherein the immune cell is selected from a lymphoid cell or myeloid cell.

Embodiment 2. The composition of Embodiment 1, wherein the immune cell comprises genetically engineered disruptions of all substantially all copies of the B2M gene.

Embodiment 3. The composition of Embodiment 1 or 2, wherein the immune cell has a loss of function of the B2M gene.

Embodiment 4. The composition of Embodiment 1-3, wherein the immune cell has a loss of function of both alleles of the B2M gene, optionally caused by contacting the immune cell with RNA encoding one or more gene-editing proteins.

Embodiment 5. The composition of any one of Embodiments 1-4, wherein the genetically engineered disruption of the B2M gene is in exon 3 of human B2M.

Embodiment 6. The composition of any one of Embodiments 1-5, wherein the genetically engineered disruption of the B2M gene is a deletion.

Embodiment 7. The composition of Embodiment 6, wherein the deletion is about 10 to about 20 nucleotides.

Embodiment 8. The composition of Embodiment 7, wherein the deletion is about 14 nucleotides.

Embodiment 9. The composition of Embodiment 7 or Embodiment 8, wherein the deletion is near nucleotides 500 to 550 of the human B2M gene.

Embodiment 10. The composition of Embodiment 9, wherein the deletion is of the sequence TTGACTTACTGAAG (SEQ ID NO: 2), or a functional equivalent thereof.

Embodiment 11. The composition of any one of Embodiments 1-10, wherein the immune cell has downregulated MHC class I expression and/or activity.

Embodiment 12. The composition of any one of Embodiments 1-11, wherein the immune cell is not substantially recognized by a host immune system upon administration to a subject.

Embodiment 13. The composition of any one of Embodiments 1-12, wherein the immune cell has reduced or eliminated susceptibility to cell killing by host T cells as compared to an immune cell which does not comprise a genetically engineered disruption in the B2M gene.

Embodiment 14. The composition of any one of Embodiments 1-13, wherein the immune cell has reduced or eliminated susceptibility to cell killing by other host immune cells as compared to another immune cell which comprises a genetically engineered disruption in the B2M gene.

Embodiment 15. The composition of any one of Embodiments 1-14, wherein the immune cell is a stealth cell.

Embodiment 16. The composition of any one of Embodiments 1-15, wherein the immune cell has reduced or eliminated host immune cell fratricide, e.g. NK-cell fratricide.

Embodiment 17. The composition of any one of Embodiments 1-16, wherein the immune cell is capable of self-activating.

Embodiment 18. The composition of Embodiment 17, wherein the immune cell is capable of self-activating in the absence of an interleukin, optionally selected from interleukin-2 (IL-2) and interleukin-15 (IL-15).

Embodiment 19. The composition of any one of Embodiments 1-18, wherein the immune cell is capable of inducing tumor cell cytotoxicity.

Embodiment 20. The composition of any one of Embodiments 1-19, wherein the immune cell is capable of inducing tumor cell cytotoxicity in the absence of an interleukin, optionally selected from IL-2 and IL-15.

Embodiment 21. The composition of any one of Embodiments 1-20, wherein the immune cell further comprises a genetically engineered disruption in a MHC II transactivator (CIITA) gene.

Embodiment 22. The composition of Embodiment 21, wherein the immune cell has downregulated MHC class II expression and/or activity.

Embodiment 23. The composition of any one of Embodiments 1-22, wherein the immune cell comprises a genetically engineered alteration in one or more genes selected from HLA-A, HLA-B, HLA-C, HLA-E, HLA-F and HLA-G.

Embodiment 24. The composition of any one of Embodiments 1-23, wherein the immune cell expresses a fusion protein comprising a B2M polypeptide and a HLA-A, HLA-B, HLA-C, HLA-E, HLA-F and HLA-G polypeptide.

Embodiment 25. The composition of Embodiment 24, wherein the fusion protein expressed by insertion of a repair template into a single or double strand break of the B2M gene; wherein the repair template comprises the coding sequence for B2M and the HLA gene.

Embodiment 26. The composition of Embodiment 24 and Embodiment 25, wherein the fusion protein replaces endogenous B2M and HLA pairs expressed by an immune cell; thereby reducing the likelihood that the immune cell will be reduced or eliminated by a host immune cell.

Embodiment 27. The composition of any one of Embodiments 1-26, wherein the immune cell does not comprise a genetically engineered alteration in one or more genes selected from HLA-A, HLA-B, HLA-C, HLA-E, HLA-F and HLA-G.

Embodiment 28. The composition of any one of Embodiments 1-27, wherein the genetically engineered alteration is a genetically engineered reduction or elimination in expression and/or activity of one or more genes selected from HLA-A, HLA-B, HLA-C, HLA-E, HLA-F and HLA-G.

Embodiment 29. The composition of any one of Embodiments 1-27, wherein the genetically engineered alteration is a genetically engineered increase in expression and/or activity of one or more genes selected from HLA-A, HLA-B, HLA-C, HLA-E, HLA-F and HLA-G.

Embodiment 30. The composition of any one of Embodiments 1-29, wherein the immune cell, optionally, an NK cell, is genetically modified to express a recombinant chimeric antigen receptor (CAR) comprising an intracellular signaling domain, a transmembrane domain, and an extracellular domain comprising an antigen binding region.

Embodiment 31. The composition of Embodiment 30, the intracellular signaling domain comprises at least one immunoreceptor tyrosine based activation motif (ITAM)-containing domain.

Embodiment 32. The composition of any one of Embodiments 30 or 31, wherein the intracellular signaling domain is from one of CD3-zeta, CD28, CD27, CD134 (OX40), and CD137 (4-1BB).

Embodiment 33. The composition of any one of Embodiments 30-32, wherein the transmembrane domain is from one of CD28 or a CD8.

Embodiment 34. The composition of any one of Embodiments 30-33, wherein the antigen binding region binds one antigen.

Embodiment 35. The composition of any one of Embodiments 30-33, wherein the antigen binding region binds two antigens.

Embodiment 36. The composition of any one of Embodiments 30-35, wherein the extracellular domain comprising an antigen binding region comprises:
a. a natural ligand or receptor, or fragment thereof, or
b. an immunoglobulin domain, optionally a single-chain variable fragment (scFv).

Embodiment 37. The composition of any one of Embodiments 30-35, wherein the extracellular domain comprising an antigen binding region comprises two of a:
a. a natural ligand or receptor, or fragment thereof, or
b. an immunoglobulin domain, optionally a single-chain variable fragment (scFv).

Embodiment 38. The composition of any one of Embodiments 30-35, wherein the extracellular domain comprising an antigen binding region comprises one of each of:
a. a natural ligand or receptor, or fragment thereof, and
b. an immunoglobulin domain, optionally a single-chain variable fragment (scFv).

Embodiment 39. The composition of any one of Embodiments 30-38, wherein the antigen binding region binds a tumor antigen.

Embodiment 40. The composition of any one of Embodiments 30-39, wherein the antigen binding region comprises one or more of:
a. CD94/NKG2a, which optionally binds HLA-E on a tumor cell;
b. CD96, which optionally binds CD155 on a tumor cell;
c. TIGIT, which optionally binds CD155 or CD112 on a tumor cell;
d. DNAM-1, which optionally binds CD155 or CD112 on a tumor cell;
e. KIR, which optionally binds HLA class I on a tumor cell;
f. NKG2D, which optionally binds NKG2D-L on a tumor cell;
g. CD16a, which optionally binds an antibody/antigen complex on a tumor cell and/or wherein the CD16a is optionally a high affinity variant, optionally homozygous or heterozygous for F158V;
h. NKp30, which optionally binds B7-H6 on a tumor cell;
i. NKp44; and
j. NKp46.

Embodiment 41. The composition of any one of Embodiments 30-40, wherein the antigen binding region comprises an immunoglobulin domain, optionally an scFv directed against HLA-E, CD155, CD112 HLA class I, NKG2D-L, or B7-H6.

Embodiment 42. The composition of any one of Embodiments 30-41, wherein the antigen binding region binds an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

Embodiment 43. The composition of any one of Embodiments 30-42, wherein the antigen binding region binds two antigen, the antigens being:
a. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2 and
b. an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v₆, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2.

Embodiment 44. The composition of any one of Embodiments 30-43, wherein the extracellular domain of the recombinant CAR comprises the extracellular domain of an NK cell activating receptor or a scFv.

Embodiment 45. The composition of any one of Embodiments 30-44, wherein the immune cell comprises a gene-edit in one or more of IL-7, CCL17, CCR4, IL-6, IL-6R, IL-12, IL-15, NKG2A, NKG2D, KIR, TRAIL, TRAC, PD1, and HPK1.

Embodiment 46. The composition of Embodiment 45, wherein the gene-edit in one or more of IL-7, CCL17, CCR4, IL-6, IL-6R, IL-12, IL-15, NKG2A, NKG2D, KIR, TRAIL, TRAC, PD1, and HPK1 is caused by contacting the cell with RNA encoding one or more gene-editing proteins.

Embodiment 47. The composition of Embodiment 46, wherein the gene-edit of causes a reduction or elimination of expression and/or activity of IL-6, NKG2A, NKG2D, KIR, TRAC, PD1, and/or HPK1.

Embodiment 48. The composition of Embodiment 46, wherein the gene-edit causes an increase of expression and/or activity of IL-7, CCL17, CCR4, IL-6R, IL-12, IL-15, and/or TRAIL.

Embodiment 49. The composition of any one of Embodiments 1-48, wherein the lymphoid cell is a T cell.

Embodiment 50. The composition of Embodiment 49, wherein the T cell is a gamma-delta T cell.

Embodiment 51. The composition of any one of Embodiments 1-48, wherein the lymphoid cell is an NK cell.

Embodiment 52. The composition of Embodiment 51, wherein the NK cell is an NK-T cell.

Embodiment 53. The composition of Embodiment 51, wherein the NK cell is a human cell.

Embodiment 54. The composition of any one of Embodiments 51-53, wherein the NK cell is derived from somatic cell of a subject.

Embodiment 55. The composition of any one of Embodiments 51-54, wherein the NK cell is derived from allogeneic or autologous cells.

Embodiment 56. The composition of any one of Embodiments 51-55, wherein the NK cell is derived from an induced pluripotent stem (iPS) cell.

Embodiment 57. The composition of Embodiment 56, wherein the iPS is derived from reprogramming a somatic cell to an iPS cell, the reprogramming comprising contacting the iPS cell with a ribonucleic acid (RNA) encoding one or more reprogramming factors, optionally selected from Oct4, Sox2, cMyc, and Klf4.

Embodiment 58. The composition of Embodiment 57, wherein the reprogramming comprising contacting the iPS cell with one or more RNAs encoding each Oct4, Sox2, cMyc, and Klf4.

Embodiment 59. The composition of any one of Embodiments 56 or 57, wherein the iPS cell is derived from allogeneic or autologous cells.

Embodiment 60. The composition of any one of Embodiments 1-59, wherein the genetically engineered disruption of the B2M comprises a gene-edit and the gene-edit is caused by contacting the cell with RNA encoding one or more gene-editing proteins.

Embodiment 61. The composition of any one of Embodiments 1-60, wherein the NK cell expresses one or more of CD56 and CD16.

Embodiment 62. The composition of Embodiment 61, wherein the NK cell expresses CD16a, which optionally binds an antibody/antigen complex on a tumor cell and/or wherein the CD16a is optionally a high affinity variant, optionally homozygous or heterozygous for F158V.

Embodiment 63. The composition of any one of Embodiments 1-62, wherein the NK cell does not express CD3.

Embodiment 64. The composition of any one of Embodiments 1-63, wherein the NK cell is CD56bright CD16dim/−.

Embodiment 65. The composition of any one of Embodiments 1-64, wherein the NK cell is CD56dim CD16+.

Embodiment 66. The composition of any one of Embodiments 1-65, wherein the NK cell is a NKtolerant cell, optionally comprising CD56bright NK cells or CD27-CD1 b-NK cells.

Embodiment 67. The composition of any one of Embodiments 1-65, wherein the NK cell is a NKcytotoxic cell, optionally comprising CD56dim NK cells or CD11b+CD27-NK cells.

Embodiment 68. The composition of any one of Embodiments 1-65, wherein the NK cell is a NKregulatory cell, optionally comprising CD56bright NK cells or CD27+NK cells.

Embodiment 69. The composition of any one of Embodiments 1-65, wherein the NK cell is a natural killer T (NKT) cell.

Embodiment 70. The composition of any one of Embodiments 1-69, wherein the NK cell secretes one or more cytokines selected from interferon-gamma (IFN-g), tumor necrosis factor-alpha (TNF-a), tumor necrosis factor-beta (TNF-b), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-Tb (MIP-1b).

Embodiment 71. The composition of any one of Embodiments 1-70, wherein the NK cell further comprises one or more recombinant genes capable of encoding a suicide gene product.

Embodiment 72. The composition of Embodiment 71, wherein the suicide gene product comprises a protein selected from the group consisting of thymidine kinase and an apoptotic signaling protein.

Embodiment 73. The composition of any one of Embodiments 60-72, wherein the gene-editing protein is selected from a nuclease, a transcription activator-like effector nuclease (TALEN), RiboSlice, a zinc-finger nuclease, a meganuclease, a nickase, a clustered regularly interspaced short palindromic repeat (CRISPR)-associated protein or a natural or engineered variant, family-member, orthologue, fragment or fusion construct thereof.

Embodiment 74. The composition of any one of Embodiments 2-73, wherein the RNA is mRNA.

Embodiment 75. The composition of Embodiment 74, wherein the RNA is modified mRNA.

Embodiment 76. The composition of Embodiment 75, wherein the modified mRNA comprises one or more non-canonical nucleotides.

Embodiment 77. The composition of Embodiment 76, wherein the non-canonical nucleotides have one or more substitutions at positions selected from the 2C, 4C, and 5C positions for a pyrimidine, or selected from the 6C, 7N and 8C positions for a purine.

Embodiment 78. The composition of any one of Embodiments 76 or 77, wherein the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

Embodiment 79. The composition of any one of Embodiments 2-78, wherein the RNA comprises a 5′ cap structure.

Embodiment 80. The composition of any one of Embodiments 2-79, wherein the RNA 5′-UTR comprises a Kozak consensus sequence.

Embodiment 81. The composition of Embodiment 80, wherein the RNA 5′-UTR comprises a sequence that increases RNA stability in vivo, and the 5′-UTR may comprise an alpha-globin or beta-globin 5′-UTR.

Embodiment 82. The composition of any one of Embodiments 2-81, wherein the RNA 3′-UTR comprises a sequence that increases RNA stability in vivo, and the 3′-UTR may comprise an alpha-globin or beta-globin 3′-UTR.

Embodiment 83. The composition of any one of Embodiments 2-82, wherein the RNA comprises a 3′ poly(A) tail.

Embodiment 84. The composition of Embodiment 83, wherein the RNA 3′ poly(A) tail is from about 20 nucleotides to about 250 nucleotides in length.

Embodiment 85. The composition of any one of Embodiments 2-84, wherein the RNA is from about 200 nucleotides to about 5000 nucleotides in length.

Embodiment 86. The composition of any one of Embodiments 2-85, wherein the RNA is prepared by in vitro transcription.

Embodiment 87. The composition of any one of Embodiments 1-86, wherein the myeloid cell is a macrophage.

Embodiment 88. The composition of Embodiment 87, wherein the macrophage is a M1 macrophage or a M2 macrophage.

Embodiment 89. A pharmaceutical composition comprising an isolated NK cell of any of the above Embodiments.

Embodiment 90. A method of making an engineered immune cell, comprising:
a. reprogramming a somatic cell to an iPS cell, the reprogramming comprising contacting the iPS cell with a ribonucleic acid (RNA) encoding one or more reprogramming factors;
b. disrupting a B2M gene in the iPS cell, the disrupting comprising gene-editing the cell by contacting the cell with RNA encoding one or more gene-editing proteins; and
c. differentiating the iPS cell into an immune cell,
d. wherein the immune cell is selected from a lymphoid cell or myeloid cell.

Embodiment 91. The method of Embodiment 90, wherein the immune cell is an NK cell.

Embodiment 92. The method of Embodiment 91, wherein the NK cell is an NK-T cell.

Embodiment 93. The method of Embodiment 91 or 92, wherein the NK cell is a human cell 94. The method of Embodiment 90, wherein the lymphoid cell is a T cell.

Embodiment 95. The method of Embodiment 94, wherein the T cell is a gamma-delta T cell.

Embodiment 96. The method of Embodiment 90, wherein the myeloid cell is a macrophage.

Embodiment 97. The method of Embodiment 96, wherein the macrophage is a M1 macrophage or a M2 macrophage.

Embodiment 98. The method of any one of Embodiments 90-97, wherein the somatic cell is a fibroblast or keratinocyte.

Embodiment 99. The method of any one of Embodiments 90-98, wherein the method provides an increased proliferation rate of iPS cells as compared to the rate of iPS cells without a disruption of the B2M gene.

Embodiment 100. The method of any one of Embodiments 90-99, wherein the method provides an increased proliferation rate of differentiating cells along a lymphoid lineage cells as compared to the rate of iPS cells without a disruption of the B2M gene.

Embodiment 101. The method of any one of Embodiments 90-100, wherein the method provides an increased expansion of differentiating cells along a lymphoid lineage cells as compared to the rate of iPS cells without a disruption of the B2M gene.

Embodiment 102. The method of any one of Embodiments 90-101, wherein the differentiating comprises embryoid body-based hematopoietic commitment.

Embodiment 103. The method of any one of Embodiments 90-102, wherein the differentiating comprises enrichment of CD34+ cells.

Embodiment 104. The method of any one of Embodiments 90-103, wherein the differentiating comprises differentiating into CD5+/CD7+ common lymphoid progenitors.

Embodiment 105. The method of any one of Embodiments 90-104, wherein the method yields CD56dim CD16+NK cells.

Embodiment 106. The method of any one of Embodiments 90-105, wherein the RNA is associated with one or more lipid selected from Table A and/or Formulae I-XVI.

Embodiment 107. The method of any one of Embodiments 90-106, wherein the immune cell is the cell of any one of Embodiments 1-86.

Embodiment 108. A method of treating cancer, comprising:
a. obtaining an isolated immune cell comprising a genetically engineered disruption in a B2M gene; and
b. administering the isolated immune cell to a subject in need thereof,
c. wherein the immune cell is a lymphoid cell or a CARmyeloid cell.

Embodiment 109. The method of Embodiment 108, wherein the immune cell is a T cell, e.g., a cytotoxic T cell or gamma-delta T cell; NK cell, e.g., a NK-T cell; or a macrophage, e.g., M1 macrophage or M2 macrophages an NK cell.

Embodiment 110. The method of any one of Embodiments 108 or 109, wherein the cancer is a blood cancer.

Embodiment 111. The method of any one of Embodiments 108 or 109, wherein the cancer is a solid tumor.

Embodiment 112. The method of any one of Embodiments 108-111, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Embodiment 113. The method of any one of Embodiments 108-112, wherein the immune cell is the cell of any one of Embodiments 1 86.

Embodiment 114. A composition comprising an isolated immune cell comprising a gene edit in a CD16a gene, wherein the immune cell is selected from a lymphoid cell or myeloid cell.

Embodiment 115. The composition of Embodiment 114, wherein the gene edit transforms the CD16a into a high affinity variant of CD16a.

Embodiment 116. The composition of Embodiment 114 or Embodiment 115, wherein the gene edit introduces a phenylalanine to valine substitution (F158V) at position 158.

Embodiment 117. The composition of Embodiment 116, wherein the cell is homozygous or heterozygous for F158V.

This invention is further illustrated by the following non-limiting examples.

### Examples

The following examples are included for illustrative purposes only and are not intended to limit the scope of the invention.

### Example 1: Cell Preparation Methods and Results

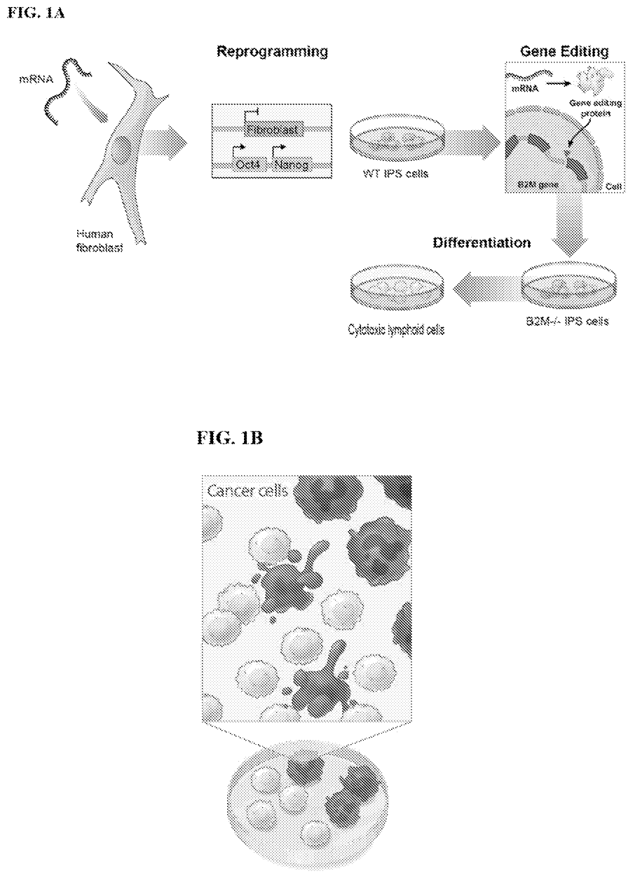

FIG. 1A shows schematic of cellular production methods used in this Example. mRNA-based cellular reprogramming (fibroblast to iPS cell) and gene-editing (beta-2-microglobulin (B2M) knockout) were employed. Further, edited cells were differentiated into cytotoxic lymphoid cells.

FIG. 1B illustrates the differentiated cytotoxic lymphoid cells killing cancer cells.

Fibroblast cells were obtained from a human subject and reprogrammed to iPS cells using an mRNA-based reprogramming.

Efficient targeting of defined loci in iPSCs using messenger RNA (mRNA)-encoded gene-editing endonucleases comprising DNA-binding domains containing novel linker region was undertaken (e.g., a gene-editing protein comprising a DNA binding domain having at least one repeat of LTPEQVVAIAS*RVD*GGKQALETVQRLLPVLCQAGHGG (SEQ ID NO: 43; the “*RVD*” corresponds to the dinucleotide “xy” of SEQ ID NO:3). Exon 3 of B2M, a key component of MHC class I molecules was targeted, and confirmed targeted editing in 10/12 lines, with 6/12 lines containing a desired biallelic deletion. Gene knockout in iPSCs was confirmed via RT-PCR and immunofluorescence in the context of B2M upregulation following exposure to interferon-γ.

More specifically, the following beta-2-microglobulin (B2M) gene was targeted:

(SEQ ID NO: 44)
AGAAATGAACTTTGAAAAGTATCTTGGGGCCAAATCATGTAGACTCTT
GAGTGATGTGTTAAGGAATGCTATGAGTGCTGAGAGGGCATCAGAAGT
CCTTGAGAGCCTCCAGAGAAAGGCTCTTAAAAATGCAGCGCAATCTCC
AGTGACAGAAGATACTGCTAGAAATCTGCTAGAAAAAAAACAAAAAA
GGCATGTATAGAGGAATTATGAGGGAAAGATACCAAGTCACGGTTTATT
CTTCAAAATGGAGGTGGCTTGTTGGGAAGGTGGAAGCTCATTTGGCCA
GAGTGGAAATGGAATTGGGAGAAATCGATGACCAAATGTAAACACTTG
GTGCCTGATATAGCTTGACACCAAGTTAGCCCCAAGTGAAATACCCTGG
CAATATTAATGTGTCTTTTCCCGATATTCCTCAGGTACTCCAAAGATTC
AGGTTTACTCACGTCATCCAGCAGAGAATGGAAAGTCAAATTTCCT
GAATTGCTATGTGTCTGGGTTTCATCCATCCGACATTGAAGTTGACTT
ACTGAAGAATGGAGAGAGAATTGAAAAAGTGGAGCATTCAGACTTG
TCTTTCAGCAAGGACTGGTCTTTCTATCTCTTGTACTACACTGAAT

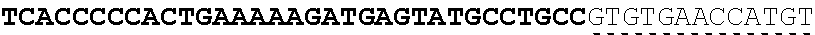

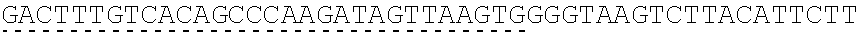

TTGTAAGCTGCTGAAAGTTGTGTATGAGTAGTCATATCATAAAGCTGCT
TTGATATAAAAAAGGTCTATGGCCATACTACCCTGAATGAGTCCCA

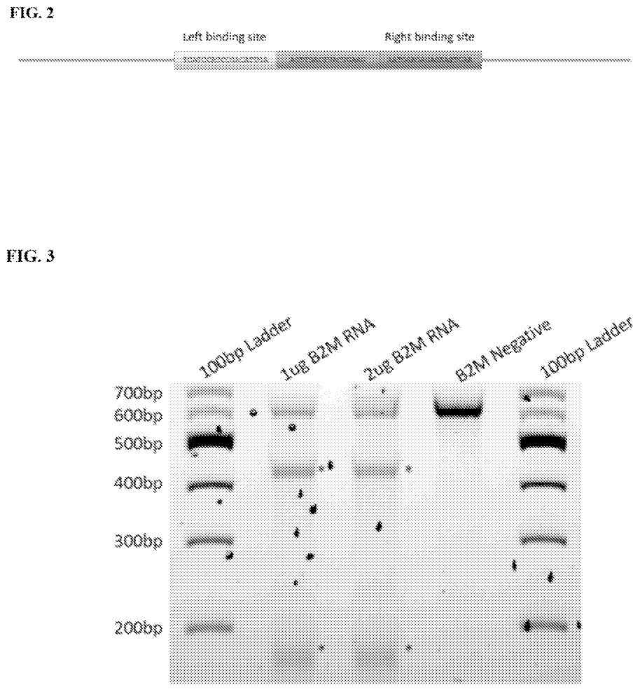

and the following primers used: B2M_Fwd:
CAGAGAAAGGCTCTTAAAAATGCAGCGCAATCTCCAG (SEQ ID NO: 45) and
B2M_Rev: CACTTAACTATCTTGGGCTGTGACAAAGTCACATGGTTCACAC (SEQ ID NO: 46) and B2M_Rev_RC:
GTGTGAACCATGTGACTTTGTCACAGCCCAAGATAGTTAAGTG (SEQ ID NO: 47). See FIG. 2.

In the above sequence, from top to bottom, the sequence features:
Bold: Exon.
Unmarked: Intron
Underlined (single): Left gene-editing protein binding site.
Underlined (double): Right gene-editing-protein binding site.
Large letters. Separation region between gene-editing-protein binding sites/cut region.
Underlined (dotted): Amplification primer binding sites.

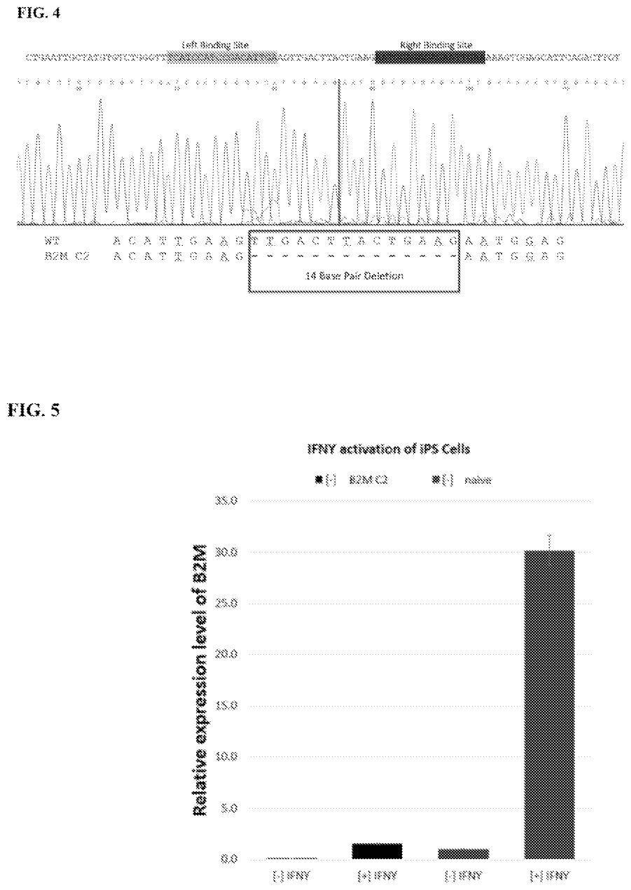

FIG. 3 shows successful gene-editing. 1.2×10⁵ iPSCs were electroporated and plated in conditioned media on a 24-well plate coated with rhLaminin-521 and grown for 48 hours. Cells were passaged into a 6-well plate coated with rhLaminin-521. Cells were cultured for an additional 4 days. Cells were split between genomic DNA extraction and 2.0×10⁴ cells were seeded into a well of a 6-well plate coated with rhLaminin-521. Amplicon length of B2M is 587 bp and edited band 1 was 416 bp (see “*”) and edited band 2 was 171 bp (see “*”). Sequencing confirmed a 14 base pair deletion in B2M (see FIG. 4).

FIG. 5 shows RNA levels of B2M with or without IFN gamma activation (“IFNY”; two left bars are the B2M knockout and the two right bars are naïve cells). 1.5×10⁴ iPSCs were plated in conditioned media on a 24-well plate coated with rhLaminin-521 and grown for 24 hours. The media was then replaced with conditioned media with or without IFN-gamma at a concentration of 25 ng/mL (t=0). Daily media changes were performed until cells were harvested at t=72 hours. RNA was extracted, quantified, and normalized for RT-qPCR analysis. The housekeeping gene was GAPDH and the tested gene was B2M.

A scalable 3D culture system for directed differentiation of human iPSCs into functional NK cells was developed. The process involved a short, embryoid body-bases hematopoietic commitment step performed either in micro-patterned wells or, in a scaled-version of the process, in multi-layer culture vessels (StemDiff™/NK Cell Kit->StemSpan T/NK differentiation kit). Hematopoietic commitment was followed by outgrowth, enrichment of CD34+ cells, and differentiation into a CD5+/CD7+ common lymphoid progenitor. The process then proceeded through a either a 14-day NK cell differentiation phase to yield functional CD56dim/CD16+NK cells or along a 22-day T cell differentiation phase to yield CD8+ T cells.

The following table outlines the cells generated:

| | | Number of | |
|---|---|---|---|
| Culture Format | | Cells into NK | Cell Counts |
| At CD34+ HSCP | Type | Differentiation | harvested |
| 3D | wild type | 1.25 × 10⁵ | 1.0 × 10³ |
| | B2M−/− | 6.25 × 10⁵ | 5.77 × 10⁵ |

This table also shows that, compared to wild type, B2M knockout cells proliferated robustly. The B2M−/−iPSC line with a 14-bp deletion at the target site (shown in FIG. 4) exhibited an increased proliferation rate both as iPSCs and during differentiation along a lymphoid lineage when compared to a wild type iPSC line.

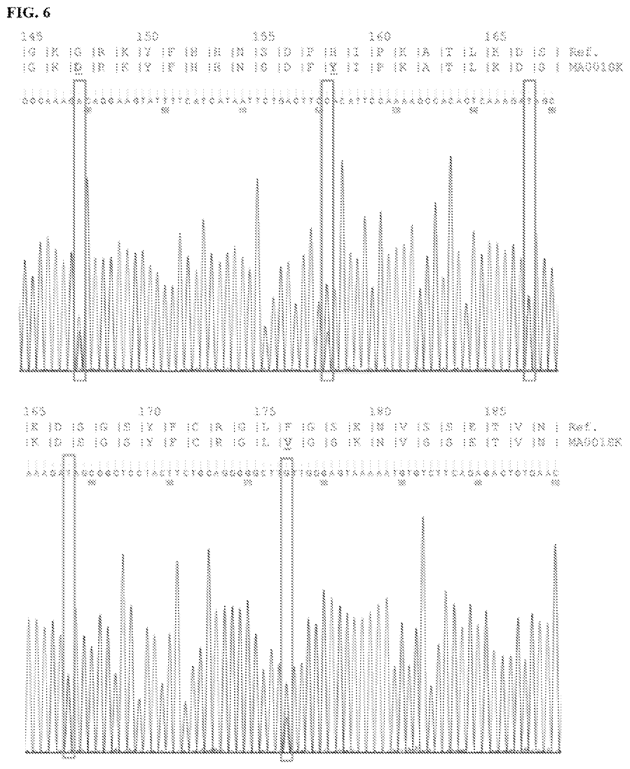

Further, resultant NK cells, as an illustrative differentiated cell type, were characterized for CD16a (UniProtKB P08637 (FCG3A_HUMAN)) and determined to be heterozygous at G147D dbSNP:rs443082, Y158H dbSNP:rs396716, and F176V dbSNP:rs396991. F176V dbSNP:rs396991 shows a higher binding capacity of IgG1, IgG3 and IgG4. See FIG. 6. gDNA was amplified with 2 step PCR: Kapa HiFi HotStart (35×/64° C. extension), primers were F: CTGATCTAGAACTTACTGTGAATCCTTGTCACCTGCCAC (SEQ ID NO: 48) and R: GATAAGAAGGAGGCCAGCACGATAGGAACATATGACAC (SEQ ID NO: 49).

This Example, inter alia, demonstrates a scalable 3D process for the differentiation of both wild-types and engineered iPSCs into functional NK cells, as an illustrative differentiated cell type. The 3D process described herein is also useful for differentiation of both wild-types and engineered iPSCs into other functional immune cells of the lymphoid or myeloid lineage, including but no limited to T cells, e.g., a cytotoxic T cells or gamma-delta T cells; NK-T cells; and macrophages, e.g., M1 macrophage or M2 macrophages. This process supports the development of next-generation cell therapies for immuno-oncology applications.

The methods disclosed herein are enhanced when transfected RNA is associated with one or more lipid selected from Table A and/or Formulae I-XVI.

### Example 2: Cells Characterization Methods and Results

Phenotypical and functional characterization assays were used to evaluate cells of Example 1.

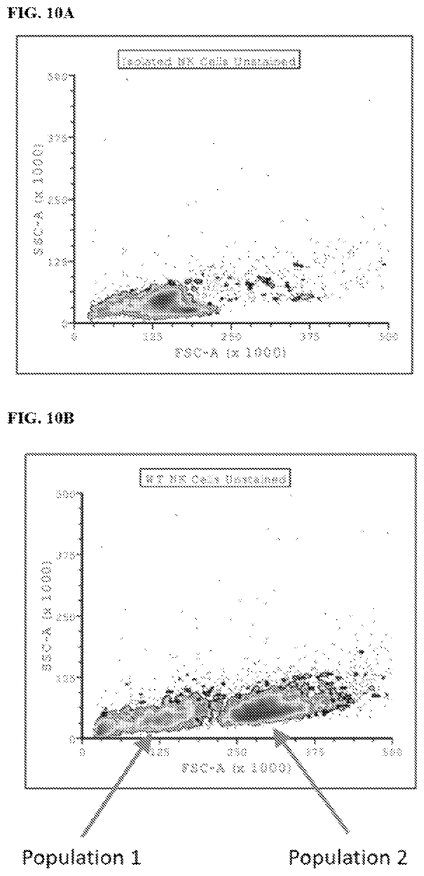

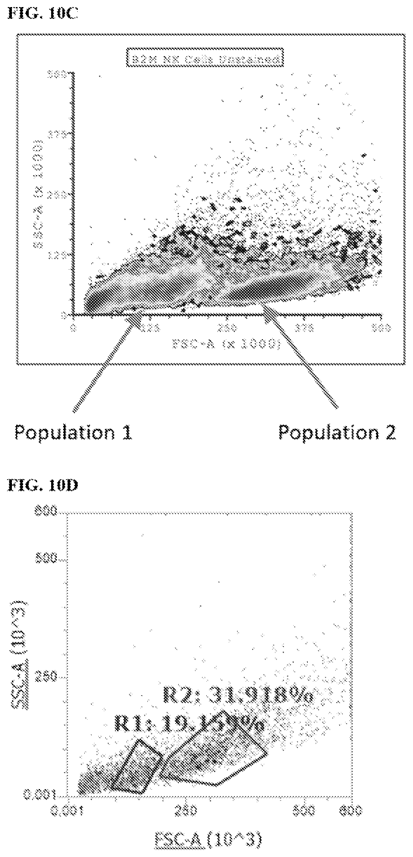

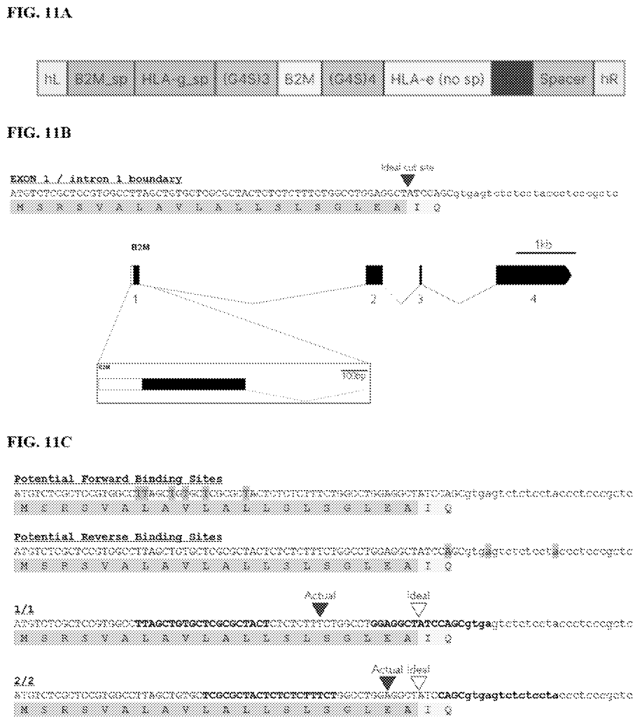

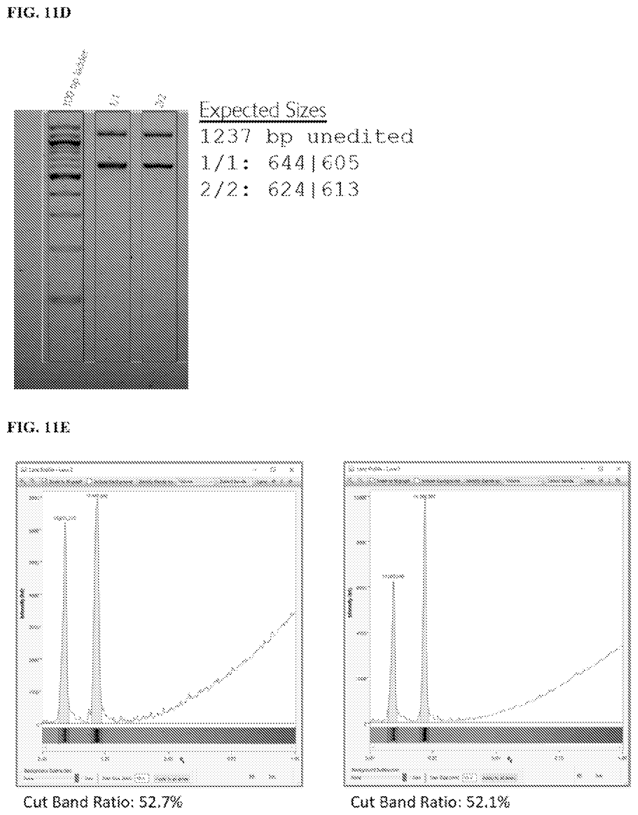

Phenotypical characterization used flow cytometry staining of surface markers, specifically CD56/CD16 (e.g., gated on CD56). CD56/NKG2D, CD56/CD45, CD56/CD3, CD56/CD244, CD56/CD94/NKG2A, CD56/NKp46, CD56/NKp44, CD56/KIRs, CD56/TRAIL, and CD56/FASL were also assessed (e.g., gated on CD56). See, FIG. 10A to FIG. 10C and the below tables:

Data in this first table characterize PBMC-Isolated NK Cells vs. iPSC-Derived NK Cells from suspension round 1:

| | | WT | | B2M^{-/-} | |
|---|---|---|---|---|---|
| | Isolated | Popula- | Popula- | Popula- | Popula- |
| | NK Cells | tion 1 | tion 2 | tion 1 | tion 2 |
| % of | | 25.6% | 74.4% | 32.9% | 67.1% |
| Population | | | | | |
| CD56 | 77.5% | 82.1% | 22.3% | 84.9% | 68.2% |
| CD16 | 16.3% | 2.06% | 0.25% | 8.23% | 21.3% |
| CD3 | 28.0% | 76.1% | 20.0% | 61.5% | 22.9% |
| CD45 | 97.8% | 99.1% | 99.4% | 99.8% | 99.8% |
| NKG2D | 9.17% | 20.9% | 59.3% | 17.0% | 43.8% |

Data in this second table characterize iPSC-Derived NK Cells—AggreWell™ vs. iPSC-Derived NK Cells from suspension round 2

| | AggreWell ™ | | | | Suspension | | | |
|---|---|---|---|---|---|---|---|---|
| | WT | | B2M^{-/-} | | WT | | B2M^{-/-} | |
| | Pop 1 | Pop 2 | Pop 1 | Pop 2 | Pop 1 | Pop 2 | Pop 1 | Pop 2 |
| CD244 | 63% | 94% | 77% | 94% | 50% | 92% | 62% | 89% |
| CD336 | 61% | 77% | 55% | 32% | 42% | 33% | 4% | 5% |
| CD3 | 70% | 89% | 65% | 14% | 65% | 44% | 46% | 14% |
| CD4 | 30% | 51% | 25% | 24% | 50% | 48% | 59% | 56% |
| TCRαβ | 1% | 9% | 49% | 9% | 6% | 3% | 55% | 49% |
| TCRγδ | 0% | 0% | 75% | 11% | 82% | 49% | 77% | 13% |

The illustrative B2M-edited, differentiated cell type, i.e., NK cells were CD45+, CD56+, CD16−, NKG2D−, KIR2DL4−, KIR2DL1−, and CD8−. See also, FIG. 10D.

Functional characterization involved measurement of cytotoxicity, activation, and evaluation of cytokine release assay, as well as a proliferation assay for an illustrative differentiated cell type, i.e., NK cells.

NK Cell cytotoxicity was measured using target cells loaded with calcien AM (a cell-permeant dye that is used to determine cell viability in most eukaryotic cells. In live cells the nonfluorescent calcein AM is converted to a green-fluorescent calcein after acetoxymethyl ester hydrolysis by intracellular esterase). Various effector (NK cell)-to-target (K-562 cell) ratios (E:T ratio) were tested to observe tumor killing. K-562 cells are a cancer cell line derived from a 53-year-old female with chronic myelogenous leukemia (CML) in terminal blast crises (greater than 30% immature cells in the bone marrow (BM), peripheral blood, a large focus of blasts in the BM, or presence of extramedullary infiltration with blast cells). These cells are commonly used for cytotoxicity assays as they lack the MHC complex required to inhibit NK activity. The experiments also include effector cells incubated with and without a cytokine cocktail (IL-15 and IL-2).

K-562 cells were loaded with calcein AM for 1 hour and washed with complete RPMI-1640 with 10% FBS prior to co-culture with NK cells. Cells were co-cultured for 18 hours with images taken every 30 minutes using the Operetta high content imager. Once a run had finished, the cell suspension was centrifuged and the media harvested. The conditioned media was tested on a Luminex MAGPIX to detect and measure the concentration of IFNg and TNFa. Cells were resuspended and stained for CD56/CD16 and CD56/CD1074a.

Activation was assessed by measuring activation marker CD107a via flow cytometry using methods as described herein and/or as well-known in the art.

For the cytokine release assay, after cytotoxicity measurements, media was harvested and cytokine release was assayed (IFNg and TNFa) using methods as described herein and/or as well-known in the art.

For proliferation, cells were incubated in the presence of activating cytokine IL-15 with media replacements occurring every three days. Every three days samples were pelleted washed, reseeded in fresh media containing the activating cytokine, and counted to trace the number of cells as well as viability over time. Cells were tracked over 28 days. During this experiment, media was saved for cytokine evaluation through a Luminex immune panel.

The remaining cells from this handling were seeded onto on untreated 96-well plate at a known cell count and images, cell counts and media changes with IL-15 occurred every 3 days for 28 days.

NK-92 cells are an interleukin-2 (IL-2) dependent natural killer cell line derived from peripheral blood mononuclear cells (PBMCs) from a 50-year-old Caucasian male with rapidly progressive non-Hodgkin's lymphoma. NK-92 cells are used as a control cell for NK cytotoxicity experiments to demonstrate the functional killing of tumor cells. NK-92 cells are used herein in the cytotoxicity assay with calcein AM. When NK cells engage, they secrete cytokines and histone into the media. Histones are highly involved in inflammation and coagulation mechanisms known as “immunothrombosis” occur, which are observable as cell “clumping”.

For cytotoxicity assay, activation, and cytokine release plate layout, samples were run in triplicate; cell mixtures were tested with and without a cytokine cocktail (IL-15+IL-2); PBMC isolated NK cells from a single donor were used as the control; PBMC isolated NK cells and 3D B2M−/− NK Cells (manufactured by methods of the present disclosure) were tested at two different E:T Ratios (20 k NK cells to 20 k K-562 Cells (1:1 E:T ratio) and 30 k NK cells to 20 k K-562 Cells (3:1 E:T ratio); 2D Wild Type and 2D B2M−/− NK Cells (manufactured by methods of the present disclosure) were tested at a 1:1 E:T ratio; and 3D Wild Type did not undergo the above testing and was plated for proliferation

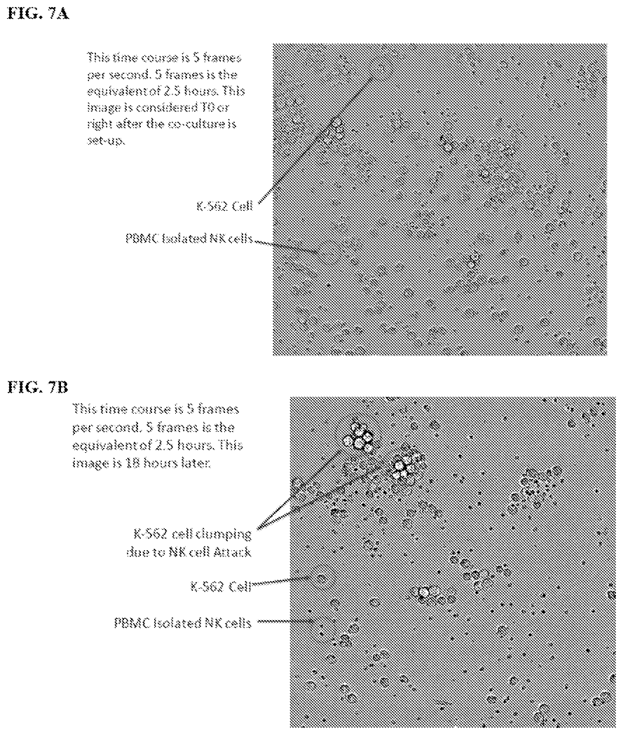

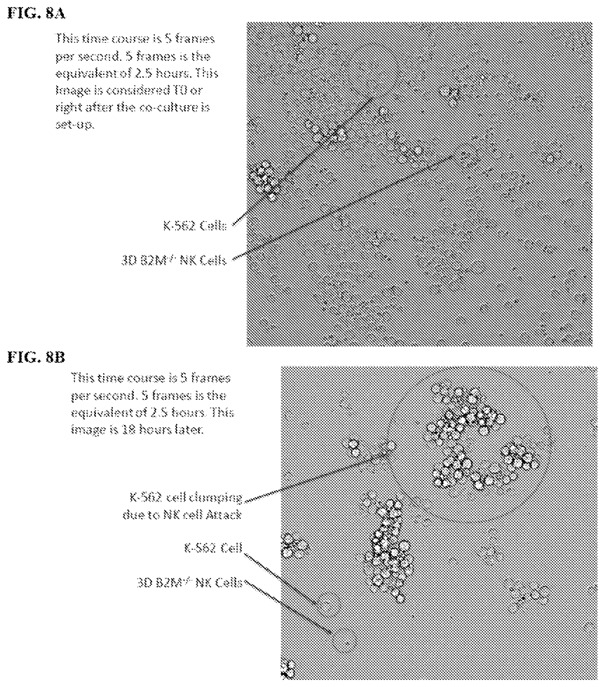

NK Cell cytotoxicity assays are shown in FIG. 7A-B and FIG. 8A-B (time course is 5 frames per second. 5 frames are the equivalent of 2.5 hours. For orientation, it is noted that K-562 cells are larger in the images herein than NK cells. FIG. 7A-B shows PBMC Isolated NK cells (i.e., control cells) co-cultured with K-562 (3:1 E:T) without cytokine cocktail at time 0 and 18 hours later. K-562 cell clumping due to NK cell attack was observed, indicating that the assay is performing as expected. FIG. 8A-B shows the 3D B2M−/− NK cells co-cultured with K-562 (3:1 E:T) without cytokine cocktail. K-562 cell clumping due to NK cell attack is observed, and at levels that are surprisingly far greater than that of the PMBC-isolated NK cells (compare FIG. 7B and FIG. 8B and note more clumping and less unengaged NK cells.

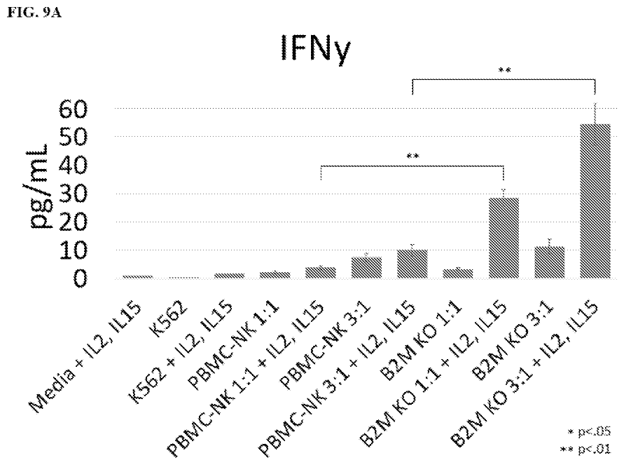

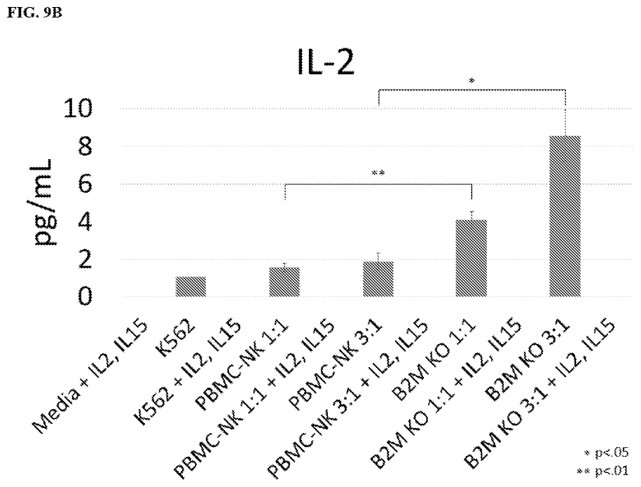

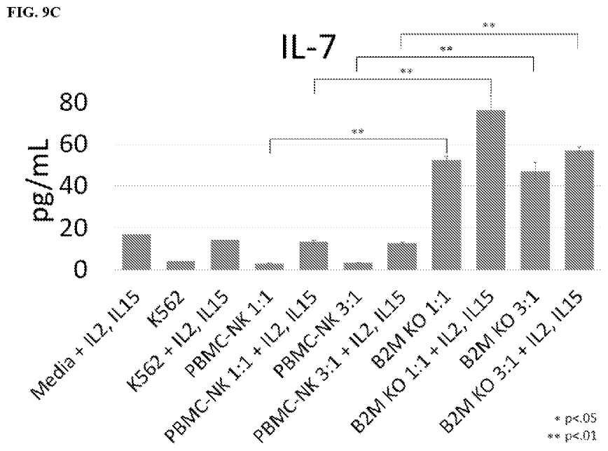

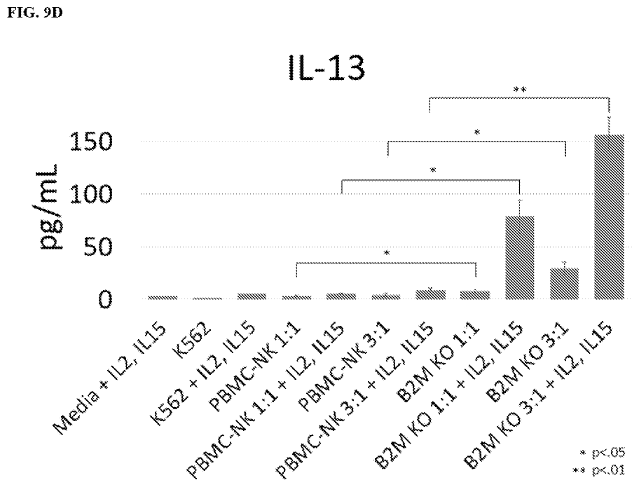

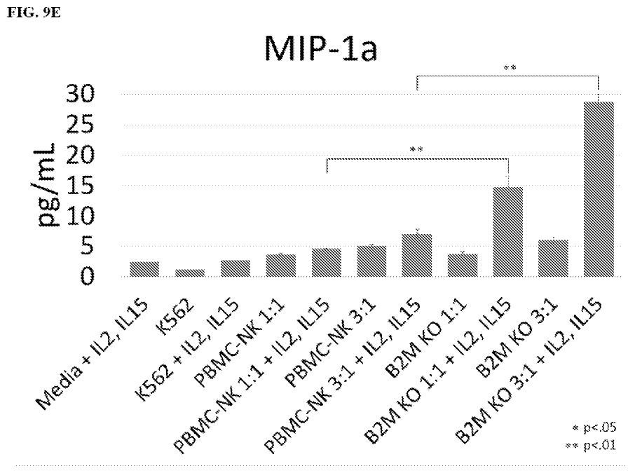

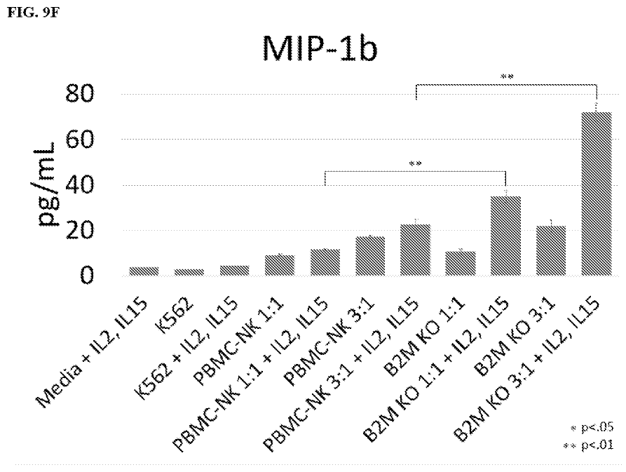

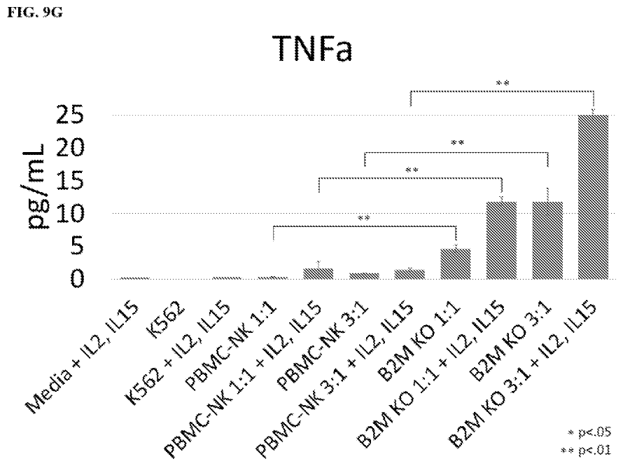

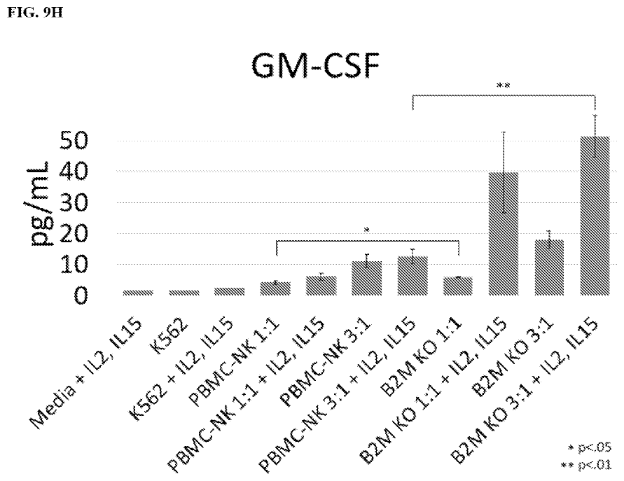

Results of the cytokine release assay with the Luminex MAGPIX are shown in FIG. 9A-FIG. 9H. Unless indicated (i.e. “+IL2, IL15”), conditions are without added IL-2 or IL-15. Further, ratio of cells are indicated (1:1 or 3:1). As elsewhere herein, wild-type PBMC-derived NK are control NK cells. FIG. 9A shows interferon gamma. FIG. 9B shows IL-2. FIG. 9C shows IL-7. FIG. 9D shows IL-13. FIG. 9E shows MIP-1a. FIG. 9F shows MIP-1b. FIG. 9G shows TNFa. FIG. 9H shows GM-CSF.

In short, and without limitation, the data herein demonstrates the generation if B2M knockout immune cells that do not self-kill but, rather, self-activate (even in the absence of cytokines like IL-2 and IL-15). Further, these B2M knockout cells can kill tumor cells (even in the absence of cytokines like IL-2 and IL-15) and have unexpected expansion and proliferation properties.

### Example 3: B2M-HLA-E Insertion

In this example, repair template (the B2M-HLA-E repair template) comprising the B2M coding sequence and the HLA-E (Major Histocompatibility Complex, Class I, E) coding sequence was inserted into a beta-2-microglobulin (B2M) edit. Here, iPSCs having their B2M gene edited, as disclosed herein, are contacted with a repair template comprising the coding sequence for HLA-E. Alternately, un-edited iPSCs are contacted with the gene-editing components to edit the B2M gene along with a repair template comprising the coding sequence for HLA-E. In both cases, the resulting cell (either as in iPSC or when differentiated into a immune cell of the lymphoid or myeloid lineage) will have an edited B2M gene and will express, in it replace, HLA-E.

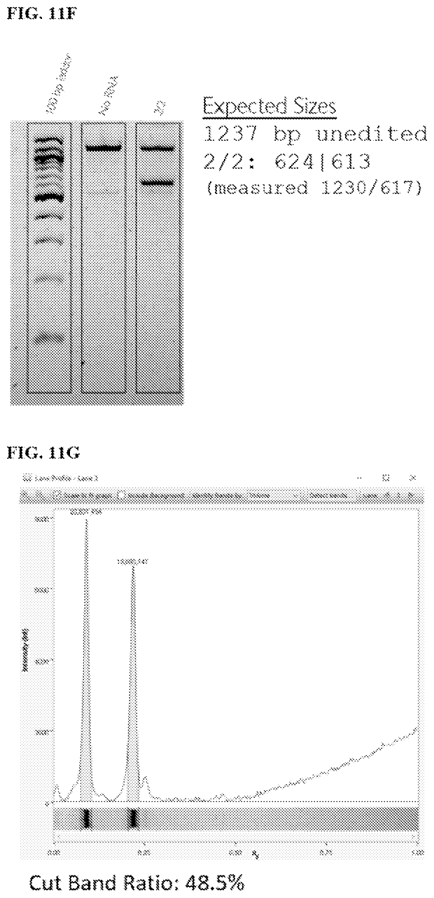

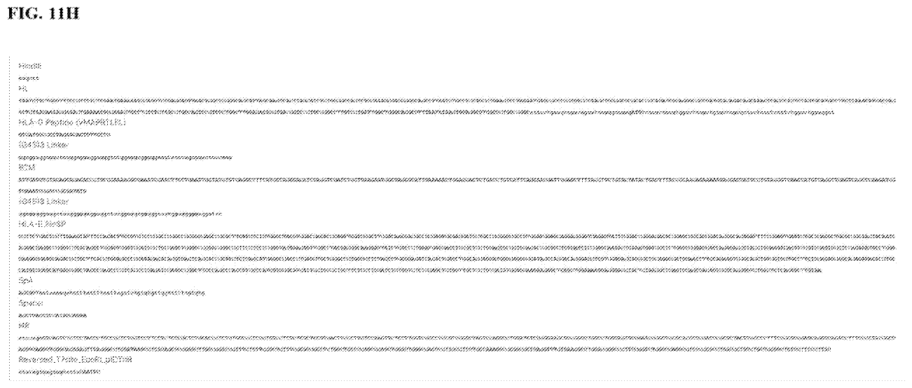

As shown in FIG. 11A the B2M signal peptide sequence (B2M_sp), which is contained entirely within Exon 1 of B2M, is included in a B2M-HLA-E repair template. Without wishing to be bound by theory, editing B2M Exon 1 and including entire B2M CDS offers the most direct path to generating the fusion.

A ideal insertion of the B2M-HLA-E repair template is located at B2M's Exon 1—Intron 1 boundary, as shown in FIG. 11B. Additional binding sites are shown in FIG. 11C, including actual lines 1/1 and 2/2 that were gene edited and which incorporated the B2M-HLA-E repair template into their genome.

Using methods disclosed herein, cells were gene edited to insert the repair template into their genome. FIG. 11D shows a gel with sizes of two lines having the B2M-HLA-E repair template (of about 1.5 kb) inserted repair template into their genome at positions 1/1 and 2/2 of FIG. 11C. In this instance, mesenchymal stem cells (MSCs) cells were gene edited. FIG. 11E shows the intensity of signal and ratios thereof from the bands shown in FIG. 11D.

Using methods disclosed herein, cells were gene edited to insert the repair template into their genome. FIG. 11F shows a gel with sizes of one line having the B2M-HLA-E repair template (of about 1.5 kb) inserted repair template into their genome at position 2/2 of FIG. 11C. In this instance, iPSCs were gene edited. FIG. 11G shows the intensity of signal and ratios thereof from the bands shown in FIG. 11F.

FIG. 11H shows relevant sequences in the B2M-HLA-E repair template.

Notably, other cells, e.g., differentiated cells as described herein, could have been gene edited and inserted with a repair template comprising a coding sequence of interest, e.g., a HLA-E coding sequence.

Through the methods of this example, the repair template causes the cell to express a B2M, e.g., as a fusion protein, with HLA-E, which needs B2M to function. Thus, this method disrupts the native, endogenous B2M gene, to prevent other HLAs from functioning, thereby “stealthing” the cells.

The methods disclosed herein are enhanced when transfected RNA is associated with one or more lipid selected from Table A and/or Formulae I-XVI.

### Example 4: High Affinity CD16a Insertion

In this example, the C16a gene is edited and replaced with the coding sequence of a high affinity CD16a variant. As shown in FIG. 12A and FIG. 12B, the phenylalanine (F) at position 158 of CD16a is targeted for gene editing such that the F is replaced with a valine (V). Relevant sequences are shown in these figures.

As shown in FIG. 12B, no NheI site appears in the amplicons for CD16a, thus with CD16NheI_ssODN_81 and CD16_NheI_ssODN_81_PT successful correction would be demonstrated by the presence of bands at ≈2127/912 bp following NheI digestion.

The methods disclosed herein are enhanced when transfected RNA is associated with one or more lipid selected from Table A and/or Formulae I-XVI.

### Example 5: Genetically Modifying a Gene-Edited and Differentiated Cell into a Chimeric Antigen Receptor (CAR)

In this example, an immune cell of the present disclosure, e.g., a T cell or NK cell, that was generated via methods of the present disclosure (e.g., by gene editing to disrupt the B2M gene and differentiation the cell from a stem cell into an immune cell of the lymphoid or myeloid lineage), is genetically modified to express a recombinant chimeric antigen receptor (CAR). The CAR comprises an intracellular signaling domain, a transmembrane domain, and an extracellular domain comprising an antigen binding region. In embodiments, the immune cell of the present disclosure is engineered to be directed to ROR1 and/or CD19. The methods for genetically modifying a cell to express a recombinant CAR are well known in the art and any such well-known method may be utilized with the immune cells of the present disclosure.

In some cases, the intracellular signaling domain of the CAR comprises at least one immunereceptor tyrosine based activation motif (ITAM)-containing domain.

In some cases, the intracellular signaling domain of the CAR is from one of CD3-zeta, CD28, CD27, CD134 (OX40), and CD137 (4-1BB).

In some cases, the transmembrane domain of the CAR is from one of CD28 or a CD8.

In some cases, the antigen binding region binds one antigen. In embodiments, the binding region binds two antigens.

In some cases, the extracellular domain comprising an antigen binding region comprises: (a) a natural ligand or receptor, or fragment thereof, or (b) an immunoglobulin domain, optionally a single-chain variable fragment (scFv). In embodiments, the extracellular domain comprising an antigen binding region comprises two of (a) a natural ligand or receptor, or fragment thereof, or (b) an immunoglobulin domain, optionally a single-chain variable fragment (scFv). In embodiments, the extracellular domain comprising an antigen binding region comprises one of each of: (a) a natural ligand or receptor, or fragment thereof, and (b) an immunoglobulin domain, optionally a single-chain variable fragment (scFv).

In some case, the antigen binding region comprises one or more of CD94/NKG2a, which optionally binds HLA-E on a tumor cell; CD96, which optionally binds CD155 on a tumor cell; TIGIT, which optionally binds CD155 or CD112 on a tumor cell; DNAM-1, which optionally binds CD155 or CD112 on a tumor cell; KIR, which optionally binds HLA class I on a tumor cell; NKG2D, which optionally binds NKG2D-L on a tumor cell; CD16a, which optionally binds an antibody/antigen complex on a tumor cell and/or wherein the CD16a is optionally a high affinity variant, optionally homozygous or heterozygous for F158V; NKp30, which optionally binds B7-H6 on a tumor cell; NKp44; and NKp46.

### Example 6: Treating Cancer

In this example, an immune cell of the present disclosure is used to treat a cancer.

The method for treating cancer comprises steps of obtaining an isolated immune cell comprising a genetically engineered disruption in a B2M gene; and administering the isolated immune cell to a subject in need thereof. In this method the immune cell is a lymphoid cell lineage or myeloid cell. In some cases, the immune cell is a T cell, e.g., a cytotoxic T cell or gamma-delta T cell; NK cell, e.g., a NK-T cell; or a macrophage, e.g., M1 macrophage or M2 macrophages an NK cell.

In some cases, additionally or alternately, the immune cell expresses a high affinity CD16a receptor.

In some cases, the immune cell expresses a fusion protein comprising a B2M polypeptide and a HLA-A, HLA-B, HLA-C, HLA-E, HLA-F and HLA-G polypeptide. The fusion protein may be expressed by insertion of a repair template into a single or double strand break of the B2M gene; in some cases, the repair template comprises the coding sequence for B2M and the HLA gene. Notably, the fusion protein replaces endogenous B2M and HLA pairs expressed by an immune cell, thereby reducing the likelihood that the immune cell will be reduced or eliminated by a host immune cell.

In some cases, the immune cell is further genetically engineered to express a chimeric antigen receptor (CAR).

The cancer may be a blood cancer.

The cancer may be a solid tumor.

The cancer may be selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1-117. (canceled)

118. A method of making an engineered immune cell, the method comprising:
(a) providing a gene-edited induced pluripotent stem (iPS) cell, wherein the gene-edited iPS cell is generated by:
(1) contacting a somatic cell with a ribonucleic acid (RNA) encoding one or more reprogramming factors, which results in reprogramming of the somatic cell into an iPS cell, and
(2) causing a disruption of a beta-2-microglobulin (B2M) gene in the iPS cell by using a synthetic RNA encoding one or more gene-editing proteins; and
(b) differentiating the gene-edited iPS cell into an immune cell, wherein the immune cell is a lymphoid cell or myeloid cell.

119. The method of claim 118, wherein the immune cell is an NK cell.

120. The method of claim 119, wherein the NK cell is an NK-T cell.

121. The method of claim 119, wherein the NK cell is a human cell

122. The method of claim 118, wherein the immune cell is a T cell.

123. The method of claim 122, wherein the T cell is a gamma-delta T cell.

124. The method of claim 118, wherein the immune cell is a macrophage.

125. The method of claim 124, wherein the macrophage is an M1 macrophage or an M2 macrophage.

126. The method of claim 118, wherein the somatic cell is a fibroblast or keratinocyte.

127. The method of claim 118, wherein the somatic cell is a human fibroblast or human keratinocyte.

128. The method of claim 118, wherein the method provides an increased proliferation rate of differentiating cells along a lymphoid lineage as compared to proliferation rate of corresponding differentiating cells generated from iPS cells without a disruption of the B2M gene.

129. The method of claim 118, wherein the method further comprises enriching for CD34+ cells.

130. The method of claim 118, wherein the differentiating comprises differentiating the immune cell into CD5+/CD7+ common lymphoid progenitors.

131. The method of claim 118, wherein the method yields CD56^{dim} CD16+ NK cells.

132. The method of claim 118, wherein the immune cell has downregulated MHC class I expression and/or activity as compared to an immune cell generated from a corresponding iPS cell without a disruption of the B2M gene.

133. The method of claim 118, wherein the immune cell is a NK cell and has reduced or eliminated NK-cell fratricide as compared to an immune cell generated from a corresponding iPS cell without a disruption of the B2M gene.

134. The method of claim 118, wherein the immune cell does not comprise a genetically engineered alteration in one or more genes selected from HLA-A, HLA-B, HLA-C, HLA-E, HLA-F, and HLA-G.

135. The method of claim 118, wherein the RNA encoding the one or more reprogramming factors is a synthetic RNA.

136. The method of claim 118, wherein the method comprises contacting the iPS cell with the synthetic RNA encoding one or more gene-editing proteins.

137. A composition comprising an isolated immune cell comprising a genetically engineered disruption in a beta-2-microglobulin (B2M) gene, wherein the immune cell is selected from a lymphoid cell or myeloid cell.
